(19) United States
(12) Patent Application Publication
(10) Pub. No.: US 2025/0075245 A1
(43) Pub. Date: Mar. 6, 2025

(54) APPARATUS AND METHOD FOR STERILIZING AND STORING DUAL MEMBRANE SENSOR COMPONENT

(71) Applicant: Broadley-James Corporation, Irvine, CA (US)

(72) Inventors: Scott T. Broadley, Laguna Beach, CA (US); Robert J. Garrahy, Laguna Niguel, CA (US); Bradley Joseph Sargent, Mission Viejo, CA (US); Michael Palmer, Ladera Ranch, CA (US)

(21) Appl. No.: 18/826,021

(22) Filed: Sep. 5, 2024

Related U.S. Application Data
(60) Provisional application No. 63/580,933, filed on Sep. 6, 2023.
(60) Provisional application No. 63/590,325, filed on Oct. 13, 2023.

(51) Int. Cl. C12Q 1/00; A61L 2/20; A61L 101/44; B65B 55/02; B65D 85/38; G01N 21/64

(52) U.S. Cl. CPC C12Q 1/006; A61L 2/206; B65B 55/02; B65D 85/38; C12Q 1/002; G01N 21/6428; G01N 21/645; A61L 2101/44; A61L 2202/181; G01N 2021/6463; G01N 2201/064

(57) ABSTRACT

Sterilizable enzymatic sensors and sensor assemblies are provided, the enzymatic sensor including at least one enzymatic sensing membrane located at or near a distal tip of the enzymatic sensor. A sensor assembly may be packaged in a partially assembled state within a flexible package, at least a portion of which is permeable to ethylene oxide (ETO). The sensor assembly may also include a flexible tube which can be connected to the enzymatic sensor such that a distal section of the enzymatic sensor is positioned within an interior of the flexible tube, and a distal tube cap connected to a distal end of the flexible tube, the cap including an internal passage allowing passage of the distal tip of the enzymatic sensor therethrough, a plurality of filtered ports, and a gasket dimensioned to form a fluid-tight seal when a portion of the sensor is inserted through the gasket. The sensor assembly may also include a sealing membrane to occlude a distal end of the internal passage of the distal tube cap. Further assembly of the sensor assembly may be performed within the flexible package after ETO sterilization.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit under 35 U.S.C. § 119 (c) of U.S. Provisional Patent Application No. 63/580,933, filed Sep. 6, 2023 and U.S. Provisional Patent Application No. 63/590,325, filed Oct. 13, 2023 the disclosure of each of which is hereby expressly incorporated by reference in its entirety.

### BACKGROUND

### Technical Field

Embodiments described herein relate to sensor systems and associated devices. In particular, embodiments described herein relate to the sterilization and storage of enzymatic sensor systems including multiple enzymatic sensing membranes.

### Description of the Related Art

In biopharmaceutical manufacturing processes, maintenance of glucose levels within the process medium is critical to the productivity and successful completion of a bioprocess. If glucose levels within the process medium are too low, the cells may metabolize all lactate produced by the cells, which can lead to the apoptosis of the cells within the process. If glucose levels are too high, the lactate produced by the cells accumulates, and steps must be taken to maintain the pH of the process medium, which in turn can increase the osmolality, or hyperosmotic stress, within the cell culture. This increase in osmolality can negatively affect the yield and other properties of the cell culture.

In an effort to maintain glucose levels of a process medium within a desired range, periodic samples may be taken of the cell culture, and these samples may be analyzed using external testing equipment. Because this is labor intensive and affects the volume of the process medium, these periodic tests are performed at wide intervals, usually once a day. The behavior of the cell process medium, and the need for glucose supplementation of the process medium, is predicted using complex algorithms in conjunction with these sporatic samplings. These predictive models have difficulty in accurately predicting the behavior of individual bioreactors, and the glucose levels can drift out of the desired range over time. In addition, errors in the sampling, modeling, or delivery of glucose can go uncorrected until after the next sample, and this delay can have a significant detrimental effect on the yield and quality of the cell culture.

### SUMMARY

In one broad aspect, an enzymatic sensor is provided, including an internal chamber, a first window in a wall of the internal chamber, a glucose-reactive membrane stack positioned over the first window, the glucose-reactive membrane stack at least partially exposed to an exterior of the enzymatic sensor, a second window in a wall of the internal chamber, a glucose-insensitive membrane stack positioned over the second window, the glucose-insensitive membrane stack at least partially exposed to the exterior of the enzymatic sensor, a light source disposed within the internal chamber and configured to illuminate the glucose-reactive membrane stack through the first window and to illuminate the glucose-insensitive membrane stack through the second window, a first sensing photodiode optically shielded from the second window and configured to detect a first fluorescence response of the glucose-reactive membrane stack to illumination from the light source, and a second sensing photodiode optically shielded from the first window and configured to detect a second fluorescence response of the glucose-insensitive membrane stack to illumination from the light source.

The first window can be formed in a first face of the sensor and the second window can be formed in a second face of the sensor, the first face oriented at an angle to the second face. The first face can be oriented generally orthogonal to the second face. Each of the first face and the second face can be oriented at an angle of roughly 45 degrees to a longitudinal axis of the sensor.

The glucose-reactive membrane stack and the first sensing photodiode can form a part of a glucose-sensing component configured to provide an indication of glucose levels in a process medium to which the sensor is exposed. The glucose-insensitive membrane stack and the second sensing photodiode can form a part of a dissolved oxygen sensing component configured to provide an indication of dissolved oxygen levels in a process medium to which the sensor is exposed.

The sensor may also include a processor configured to provide an output signal indicative of glucose concentration in a process medium to which the sensor is exposed. The processor can be configured to determine the glucose concentration based at least in part on a difference between an indication of oxygen concentration at the glucose-reactive membrane stack and an indication of oxygen concentration at the glucose-insensitive membrane stack. The processor can be further configured to determine the glucose concentration based at least in part on a measured temperature of the process medium to which the sensor is exposed. The processor can be further configured to determine the glucose concentration based at least in part on an absolute measurement of the dissolved oxygen levels in the process medium to which the sensor is exposed.

The glucose-reactive membrane stack can include glucose oxidase. The sensor can additionally include a rate-limiting membrane disposed on an outer surface of the glucose-reactive membrane stack. The rate-limiting membrane can include an oxygen-permeable material including a plurality of holes extending therethrough to control glucose diffusion through the rate-limiting membrane. The sensor of can additionally include a rate-limiting membrane disposed on an outer surface of the glucose-insensitive membrane.

Each of the first and second sensing photodiodes can be shielded by a filter which permits the fluorescent responses to pass therethrough which filtering any illumination from the light source. The internal chamber can include a hermetically sealed chamber,

The glucose-reactive membrane stack can include a first dissolved oxygen sensing membrane positioned over the first window and a glucose-reactive membrane positioned over the first dissolved oxygen sensing membrane, and the dissolved oxygen sensing membrane can provide a fluorescence response indicative of the dissolved oxygen at the first dissolved oxygen sensing membrane. The glucose-reactive membrane stack can include a rate-limiting membrane disposed on an outer surface of the glucose-reactive membrane, the rate-limiting membrane including an oxygen-permeable material including a plurality of holes extending therethrough to control glucose diffusion through the rate-limiting membrane. The glucose-insensitive membrane stack can include a second dissolved oxygen sensing membrane positioned over the second window and a glucose-insensitive membrane positioned over the second dissolved oxygen sensing membrane, and the second dissolved oxygen sensing membrane can provide a fluorescence response indicative of the dissolved oxygen at the second dissolved oxygen sensing membrane.

In another broad aspect, a packaged sensor assembly is provided, including a flexible package, at least a portion of the flexible package being permeable to ethylene oxide (ETO), and a partially-assembled sensor assembly disposed within the flexible package, the partially-assembled sensor assembly including an enzymatic sensor including at least one enzymatic sensing membrane located at or near a distal tip of the enzymatic sensor, a flexible tube, the flexible tube configured to be securely connected at a proximal end to the enzymatic sensor such that a distal section of the enzymatic sensor is positioned within an interior of the flexible tube, a distal tube cap connected to a distal end of the flexible tube, the distal tube cap including an internal passage dimensioned to allow passage of the distal tip of the enzymatic sensor therethrough, a plurality of filtered ports in fluid communication with the internal passage, and a gasket at or near a proximal end of the internal passage, the gasket dimensioned to form a fluid-tight seal when a portion of the distal section of the enzymatic sensor is inserted through the gasket, and a sealing membrane dimensioned to occlude a distal end of the internal passage of the distal tube cap.

The partially-assembled sensor assembly disposed within the flexible package can additionally include an aseptic connector configured to be positioned on a side of the sealing membrane opposite the distal tube cap to retain the sealing membrane in place between the distal tube cap and the aseptic connector. The package can additionally include a clamp configured to clamp the aseptic connector to the distal tube cap.

The enzymatic sensor can include a glucose-reactive membrane stack, and a glucose-insensitive membrane stack. The enzymatic sensor can be configured to provide an indication of a glucose concentration in a process medium to which the enzymatic sensor is exposed based at least in part on fluorescent responses of the glucose-reactive membrane stack and the glucose-insensitive membrane stack to illumination from a common light source. The enzymatic sensor can include any of the embodiments of enzymatic sensors described herein.

In another broad aspect, a method of sterilizing a sensor assembly is provided, the method including providing any of the embodiments of packaged sensor assemblies described herein, exposing the package to ethylene oxide (ETO), removing the ETO from the package, and while the package remains intact, performing an initial assembly process which results in the sealing membrane being secured in place to occlude the distal end of the internal passage of the distal tube cap, the interior of the flexible tube being sealed against unfiltered airflow into or out of the interior of the flexible tube, and the enzymatic sensor being positioned within the flexible tube.

The method can additionally include clamping an aseptic connector to a distal end of the distal tube cap, where the sealing membrane is disposed between the distal tube cap and the aseptic connector. The method can additionally include, after performing the initial assembly process, removing the sensor assembly from the flexible package, advancing a sensor tip of the enzymatic sensor assembly into the internal passage of the distal tube cap such that the enzymatic membranes of the sensor tip are positioned within the internal passage, and the gasket cooperates with a section of the enzymatic sensor to form a proximal end of an internal storage chamber, and filling, using the filtered ports, the internal storage chamber with a storage solution. The method can additionally include mechanically restraining movement of the sensor tip using a restraining clip secured to the sensor assembly.

In another broad aspect, a method of monitoring a glucose concentration of a process medium, is provided, the method including providing a sterilized enzymatic sensor assembly, the sensor assembly including a sensor including a sensor tip, the sensor tip including at least one enzymatic sensing membrane, the sensor tip stored within an internal storage chamber defined at its distal end by a sealing membrane, and advancing the sensor tip through the sealing membrane and into a bioprocess container to expose the at least one enzymatic sensing membrane to a process medium within the bioprocess container.

The method can additionally include forming an aseptic connection between an aseptic connector on the distal end of the sterilized enzymatic sensor assembly and an aseptic connector on a port in a wall of the bioprocess container prior to advancing the sensor tip through the sealing membrane, where the sensor tip is advanced through the aseptic connection formed using the aseptic connectors.

In another broad aspect, a sterilized enzymatic sensor assembly is provided, including any of the embodiments of enzymatic sensors described herein, the glucose-reactive membrane stack and the glucose-insensitive membrane stack located in a sensor tip at or near a distal end of the enzymatic sensor, a flexible tube, the flexible tube securely connected at a proximal end to the enzymatic sensor such that a section of the enzymatic sensor extends through the interior of the flexible tube, a distal tube cap connected to a distal end of the flexible tube, the distal tube cap including an internal passage dimensioned to allow passage of the distal tip of the enzymatic sensor therethrough, a plurality of filtered ports in fluid communication with the internal passage, and a gasket at or near a proximal end of the internal passage, the gasket forming a fluid-tight seal with a portion of the distal section of the enzymatic sensor extending through the gasket, a sealing membrane occluding a distal end of the internal passage of the distal tube cap, the sealing membrane and the fluid-tight seal defining an internal storage chamber within the internal passage of the distal tube cap, and a storage solution within the internal storage chamber to provide wet storage for the glucose-reactive membrane stack and the glucose-insensitive membrane stack.

### BRIEF DESCRIPTION OF THE DRAWINGS

FIG. 1A is a perspective view of a sensor tip having two sensing surfaces.

FIG. 1B is another perspective view of the sensor tip of FIG. 1A.

FIG. 2 is an exploded assembly view of the sensor tip of FIG. 1A.

FIG. 3 is a perspective view of a front-end electronics assembly configured for use with the dual membrane sensor tip 100 of FIG. 1A.

FIG. 4A is a perspective view of the front-end electronics assembly of FIG. 3, shown relative to an internal mask.

FIG. 4B is another perspective view of the front-end electronics assembly of FIG. 3, shown relative to an internal mask.

FIG. 5A is a perspective cross-sectional view of the assembled sensor tip of FIG. 1A.

FIG. 5B is a side cross-sectional view of the assembled sensor tip of FIG. 5A illustrating the redirection of light within the sensor tip during operation.

FIG. 6A is a top view illustrating a dual-membrane enzymatic sensor comprising the sensor tip of FIG. 1A.

FIG. 6B is a perspective view illustrating the dual-membrane enzymatic sensor of FIG. 6A.

FIG. 6C is another perspective view illustrating a distal portion of the dual-membrane enzymatic sensor of FIG. 6A.

FIG. 7 is a perspective exploded view illustrating various components of a sterilizable enzymatic sensor insertion assembly comprising a dual-membrane enzymatic sensor such as the dual-membrane enzymatic sensor of FIG. 6A, comprising a sensor tip such as the sensor tip of FIG. 1A.

FIG. 8A is a perspective exploded view illustrating various components of the sterilizable enzymatic sensor assembly of FIG. 7, in which a distal section of the insertion assembly is shown in an assembled state.

FIG. 8B is a perspective exploded view illustrating the components of the sterilizable enzymatic sensor assembly as shown in FIG. 8A, with the sensor inserted into the distal section of the insertion assembly.

FIG. 9 is a flow diagram depicting certain steps in a series of processes including sterilizing an enzymatic sensor assembly, preparing the sterilized enzymatic sensor assembly for storage, and inserting the sterilized enzymatic sensor assembly into a bioprocess container for use in measuring a property of a medium within the bioprocess container.

FIG. 10A is a perspective view of the partially assembled sensor assembly, disposed inside a sterilization package which includes a material permeable to a sterilization chemical.

FIG. 10B is an alternative illustration of the partially assembled sensor assembly of FIG. 10A.

FIG. 10C is a perspective view of the sensor assembly of FIG. 10A, shown in an assembled state within the sterilization package.

FIG. 11A is a perspective view of the sensor assembly of FIG. 10A in an assembled state.

FIG. 11B is a side partial cross-sectional view of the assembled sensor assembly, in which the sensor assembly is shown in a cross-section along the section line A-A of FIG. 11A to display the sensor disposed within the sensor assembly.

FIG. 11C is a perspective partial cross-sectional view of the assembled sensor assembly of FIG. 11B.

FIG. 11D is another perspective partial cross-sectional view of the assembled sensor assembly of FIG. 11B.

FIG. 11E is a detail view of a portion of the sensor assembly of FIG. 11B including the distal sensor cap and the aseptic connector.

FIG. 12A is a side view of the sensor assembly of FIG. 11A, in which the sensor has been advanced to a distal position in preparation for storage.

FIG. 12B is a side partial cross-sectional view of the sensor assembly of FIG. 12A, in which the sensor assembly is shown in a cross-section along the section line B-B of FIG. 12A to display the sensor disposed within the sensor assembly.

FIG. 13A is a perspective view of a sensor assembly such as the sensor assembly of FIG. 12A, with a retaining clip depicted adjacent the sensor assembly.

FIG. 13B is a perspective view of a sensor assembly and retaining clip of FIG. 13A, with the retaining clip installed on the sensor assembly.

FIG. 13C is a perspective view of a sensor assembly and retaining clip of FIG. 13A, with the sensor assembly is shown in a partial cross-section.

FIG. 14 is a perspective view of the sensor assembly of FIG. 12A, shown being connected to a port in a wall of a bioprocess container.

FIG. 15A is a perspective view of a sensor assembly similar to the sensor assembly of FIG. 14, with the sensor having been advanced to a measurement position in which the sensor tip of the sensor extends into a bioprocess container, shown in partial cross-section.

FIG. 15B is a top plan view of the sensor assembly of FIG. 15A.

FIG. 15C is a side partial cross-sectional view of the sensor assembly of FIG. 15A, in which the sensor assembly is shown in a cross-section along the section line C-C of FIG. 15B to display the sensor disposed within the sensor assembly.

### DETAILED DESCRIPTION

A bioprocess can include an initial stage primarily devoted to cell growth, and a later stage at which the cells are producing a desired product. In such a bioprocess, such as an exemplary 15-day cell culture bioreactor run, roughly the first half of the run may be primarily devoted to cell growth within a basal media, and the latter half primarily devoted to generation of a desired product using the grown cells.

Glucose is the primary energy source for mammalian cells grown in bioreactors. In many bioprocesses, a large amount of glucose is introduced to the basal media at the onset of the bioprocess. This initial glucose supply, which can be in the range of 6 to 8 g/L, fuels the initial cell growth. As growth continues, glucose is consumed, and the cells produce lactate as a metabolism byproduct. If the glucose remains or is maintained at a level which is not rate-limiting, lactate continues to be produced in large amounts, and will accumulate in the cell culture. Among other effects, this lactate accumulation will alter the pH of the cell culture, necessitating the addition of a base solution such as diluted sodium carbonate (Na2CO3) to the cell culture to maintain the pH within the desired range. In some bioprocesses, the desired pH range may be between 6.9 and 7.1.

Cell culture media, such as basal cell culture media and feed cell culture media, are prepared with a final osmolality in the range of 270-330 mOsm/Kg. The addition of base solution to the cell culture to counteract the pH shift due to lactate accumulation has a side effect of increasing the osmolality of the cell culture, and this increase in hyperosmotic stress can affect the cell culture in negative ways.

If the osmolality of the cell culture media becomes too high, such as a level greater than 400 mOsm/Kg, this osmolality increase can decrease the viability of the cell culture and decrease production yield. In addition, the osmolality of the cell culture can affect critical quality attributes of the products, such as proteins which may not fold correctly if they are not glycosylated properly. The effect of lactate accumulation can be particularly pronounced at the end of the bioprocess, as the lactate accumulation can cause production of the desired product in the final portion of the run to taper off due to the less favorable conditions.

Lactate accumulation can be controlled if the glucose levels of the cell culture are constrained to a desired range, which can be at or near a range of 0.5 to 1.5 g/L once the initial glucose supply is consumed. When the glucose levels are maintained within a suitable range, the mammalian cells consume glucose more slowly, resulting in a reduction in lactate production. When glucose levels are maintained at a rate-limiting level, cells will also consume the lactate byproduct, further controlling lactate accumulation.

Insufficient glucose supply can also negatively affect a bioprocess. If the available glucose is fully consumed, the cells will quickly consume the remainder of the lactate. Once both the glucose and lactate are consumed, the cells are left without an energy source, and apoptosis, or programmed cell death, will be irreversibly triggered, killing the cells and putting an end to production of the desired product.

When the glucose levels are controlled such that the cells are consuming a combination of both glucose and lactate, the lactate levels can be maintained at a suitably low level, such as below 2 g/L or below 1 g/L. This can reduce or even eliminate the need to add a base solution to control the pH level of the cell culture. In turn, osmolality levels remain low throughout the duration of the bioprocess, significantly improving overall cell culture performance, both in terms of the overall output and the quality of the produced product.

Because the enzymes in glucose sensors are severely damaged by autoclaving or gamma sterilization methods, measurements of glucose during an active bioprocess within a bioreactor process medium are typically performed by periodically removing samples of the cell culture and measuring the glucose concentration using separate equipment. Because this is labor intensive, and because it requires the permanent removal of a volume of the cell culture, these manual grab samples are performed at wide intervals, often one per day. In addition, the manual grab sampling and separate testing introduces additional delay in the ability to measure and compensate for the glucose readings.

Because of the long periods between measurements, an algorithmic model may be used to predict the glucose consumption rate over the course of the period between samples. Glucose may be supplied to the bioprocess during the periods between measurements in an attempt to maintain the glucose levels within the desired range and reduce the need for pH correction via addition of base solutions. It is difficult, however, to accurately predict the behavior of the cell culture, given variances between individual bioreactors and cell cultures, as well as the large number of factors that can affect glucose consumption. Even with such predictive glucose addition, the increase in osmolality over the course of the bioprocess can reduce the overall yield of the bioprocess, as lactate accumulation can occur during periods when the glucose level increases beyond the upper end of the desired range.

In some embodiments, continuous or on-demand monitoring of glucose levels within a bioprocess can be used to maintain glucose levels within a process medium at a desired level or within a desired range for the duration of a bioprocess. If the monitoring of glucose levels can be automated and done without removal of samples of the cell culture, the measurement frequency can be increased without significant labor or yield costs.

By more frequency or continuously monitoring glucose levels, the glucose levels may be maintained within a desired range. The range within which the glucose levels can be maintained using continuous glucose measurement may be narrower than the glucose ranges which may be maintained with predictive glucose supply and long periods between manual measurements. This may eliminate the need for pH adjustment, and the avoidance of the corresponding reduction in osmolality can significantly increase the volume and quality of the bioprocess product.

Many methods for the detection of glucose are centered around the use of enzymes to catalyze the reaction of glucose and oxygen to produce gluconic acid and hydrogen peroxide. The enzymes glucose oxidase and catalase promote the following reactions: In a first reaction, Glucose+O₂+H₂O→gluconic acid+H₂O₂. In a second reaction, H₂O₂→½ O₂+H₂O. The overall reaction is thus: Glucose+½ O₂→gluconic acid.

Detection of glucose is usually done by either measuring the intermediate production of H₂O₂ or by the depletion of oxygen. Hydrogen peroxide detection suffers from interfering reactions at the exposed electrode surface leading to signal drift. Detection of oxygen can be accomplished through a silicone rubber membrane to protect the sensing surface. In either case, the oxygen concentration is likely to be significantly less than the glucose concentration causing the above reactions to be rate limited by the oxygen and insensitive to changes in glucose. This oxygen deficit can be overcome by controlling the relative rates of glucose and oxygen diffusion to the glucose oxidase membrane.

Using oxygen for the measurement, the glucose concentration is then a function of the difference between the oxygen sensor with a glucose oxidase membrane and a reference oxygen sensor without the enzyme. In some embodiments, both the glucose oxidase mediated sensor and the reference sensor are combined into the same probe to avoid issues with local variations in the oxygen level.

In some embodiments, glucose measurements may be performed by exposing a membrane containing glucose oxidase (GOx), and measuring the amount of oxygen (O2) generated during the resultant reaction. However, the amount of oxygen generated by exposure of the GOx membrane to a process medium is dependent on both the glucose level of the process medium, and the oxygen saturation of the process medium. The oxygen saturation of a process medium during a bioprocess can be substantially less than 100%, in some embodiments on the order of 40%. Without information regarding the oxygen saturation of the process media, a glucose sensor cannot compensate for the oxygen saturation to achieve a precise measurement.

In some embodiments, two separate sensors may be used to obtain an accurate measurement of glucose: a glucose sensor and a dissolved oxygen (DO) sensor. When both sensors are exposed to the process medium, the output of the DO sensor can be used to compensate for the oxygen saturation and obtain an accurate measurement for the glucose level of the process medium. However, the use of multiple sensors increases the complexity of the system.

For highest precision and accuracy, an in situ glucose sensor response is compared with an in situ dissolved oxygen sensor. The difference between the two sensors can be correlated to glucose concentration in the media. However, if the glucose sensor responds more slowly than the dissolved oxygen sensor then the calculations to determine the correlation between the two sensors will suffer in precision and accuracy.

Furthermore if the glucose concentration is determined with two separate sensors, a glucose sensor and a DO sensor, the placement in the wall of the bioreactor can be problematic. The media in the bioreactor can be agitated to keep the aeration bubbles and cell distribution as even as possible throughout the bioreactor. Despite this effort the media can become stratified with cells accumulating in higher densities in the lower portion of the bioreactor. This can result in the DO sensor being in a media region of different dissolved oxygen partial pressure than the glucose sensor. This can result in the loss of a degree of precision and accuracy in the calculations of the glucose concentration in the bioreactor media.

In other embodiments, a single sensor device can include both a DO sensing component providing an indication of the oxygen saturation in the process medium, as well as a glucose sensing component providing an indication of the glucose levels in the process medium. Because these sensor components can be essentially co-located, they are exposed to the same portions of the process medium, and stratification of the process medium will not result in output variance resulting for differences in the dissolved oxygen concentration in the immediate vicinities of the respective sensors.

In some embodiments, the differential between the two sensor outputs can be converted directly to an oxygen-compensated glucose measurement. With proper selection of the thicknesses of an outer membrane of each sensor, dissolved oxygen and glucose diffusion rates can be adjusted and tailored to create glucose and DO sensing surfaces that respond identically to changes in the oxygen partial pressure of the media.

In some embodiments, the use of optical technology for both the glucose sensing surface and the DO sensing surface enables the sensor to be hermetically sealed, and therefore sterilizable by ETO gas. All measurements can be made through optical windows that can be hermetically sealed to the metal sensor body. This prevents the highly toxic ETO gas from entering the sensor where it would be difficult to remove by vacuum.

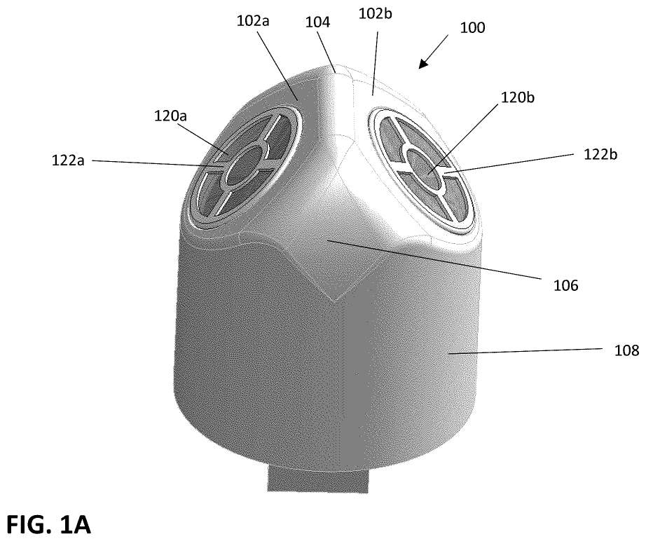

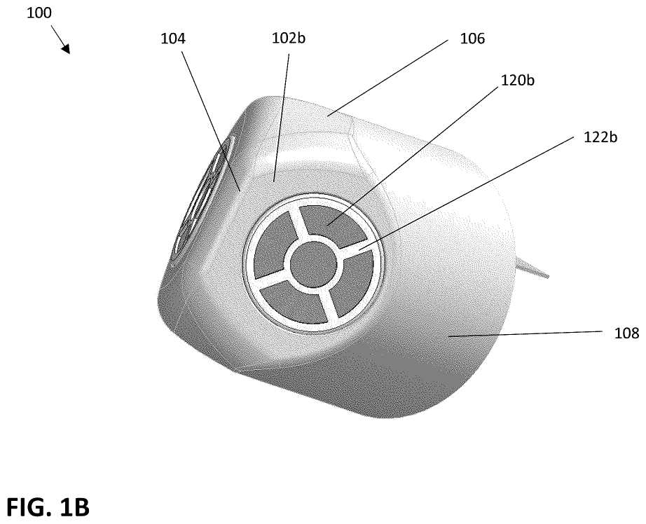

FIG. 1A is a perspective view of a sensor tip having two sensing surfaces. FIG. 1B is another perspective view of the sensor tip of FIG. 1A. In some embodiments, the two sensing surfaces can be used by a glucose sensor which compensates for variances in oxygen saturation to provide an accurate glucose measurement using only a single probe. The sensor tip 100 has first and second sensing surfaces 120a and 120b. First sensing surface 120a is located on a first major face 102a of the angled proximal end of the tip 100, and second sensing surface 120b is located on a second major face 102b of the angled distal end of the sensor tip 100. In one embodiment, each of first major face 102a and second major face 102b are located at a 45-degree angle to a longitudinal axis of a sensor having the sensor tip 100 at its distal end, although a wide variety of other possible arrangements can be used as well. The sensing surfaces 120a and 120b may be the exposed upper surfaces of a layer or stack of layers, as discussed in greater detail below. In one embodiment, the sensor tip can comprise a material such as stainless steel, although other suitable materials may also be used.

The sensor tip 100 can form a part of an enzymatic sensor assembly that is capable, with proper design of packaging and a sensor insertion structure, of being sterilized using ethylene oxide (ETO) and stored within a sterile package after sterilization, as described in greater detail below. The sensor insertion structure can then allow the sensor assembly to be inserted in a sterile fashion into a bioprocess container such as a bioreactor without contaminating the contents of the bioprocess container.

The first and second major faces 102a and 102b of the angled distal end of the sensor tip are located at an angle to one another, on opposite side of a rounded vertex 104 extending between the two. Proximal the angled major faces 102a and 102b, the sensor tip includes a generally cylindrical portion 108. The sensor tip 100 has a rounded transition between the generally cylindrical portion 108 and the proximal edges of the major faces 102a and 102b and the minor faces 106. In the illustrated embodiment, the major faces 102a and 102b are generally orthogonal to one another, although other orientations, shapes, and numbers of faces may be used in other sensor designs, to accommodate greater or fewer numbers of sensing surfaces.

In the illustrated embodiment, the first and second sensing surfaces 120a and 120b are located closer to the proximal side of the major faces 102a and 102b than to the rounded vertex 104 at the distal tip of the sensor tip 100. Such an arrangement can provide additional protection for the sensing surfaces 120a and 120b, reducing the likelihood of mechanical interference or damage to the sensing surfaces 120a and 120b if the distal tip of the sensor contacts another object. In addition, each of the sensing surfaces 120a and 120b. Retaining structures 122a and 122b having apertures formed therein overlie the sensing surfaces 120a and 120b, holding in place the underlying layers and providing additional protection from mechanical interference or damage to those layers.

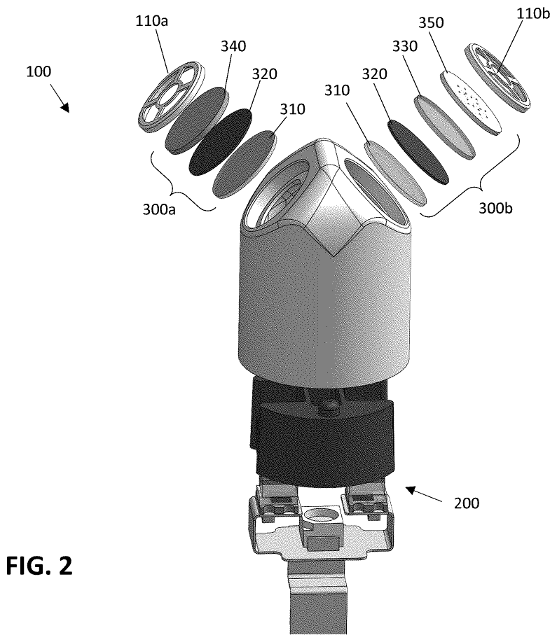

FIG. 2 is an exploded assembly view of the sensor tip 100. It can be seen in FIG. 2 that the sensing surface 120b is the exposed outer surface of a membrane stack 300b which includes a glass window 310, an O₂-sensitive membrane 320, and a glucose oxidase-containing (GOx-containing) membrane 330, and the membrane stack is retained in place within a cavity or depression in the major face 102b of the sensor tip 100. In some embodiments, a separate rate-limiting membrane 350 may also be included in the membrane stack 300b.

An aperture at the base of the cavity exposes the membrane stack 300b to the interior of the sensor tip 100. Because the cross-sectional size of the aperture is smaller than a cross-sectional size of the glass window 310, an inwardly extending lip at the bottom of the cavity cooperates with the membrane retainer 110b to hold the membrane stack 300b.

A membrane stack such as the membrane stack 300b can be illuminated by an LED and the reflected light measured using a photodiode, in a process in which luminescent detection of oxygen provides an indication of glucose levels in a medium to which the sensing surface 120b is exposed, as described in greater detail below. However, the resultant current through a photodiode is also a function of the oxygen saturation of the medium in contact with the sensing surface 120b. In some embodiments, such a glucose sensor can be used in conjunction with a separate dissolved oxygen sensor to provide an O₂-compensated absolute glucose measurement.

In other embodiments, however, the membrane stack 300b can be used in conjunction with the membrane stack 300a to compensate for the oxygen saturation and provide an accurate glucose measurement using only a single sensor. Like the membrane stack 300b, the membrane stack 300a also includes a glass window 310 and an O₂-sensitive membrane 320. However, the membrane 340 at the outer surface of the membrane stack 300a does not contain GOx. In some embodiments, the membrane 340 may share at least certain properties with the membrane 330, other than the inclusion of GOx. By using membranes which have particular properties, the membrane 340 may serve as a rate limiter or otherwise adjust a response time of the membrane stack 300a to be similar to that of the membrane stack 300b. This can provide, for example, more stable differential measurements when a sensor assembly including the sensor tip 100 begins operation. In some other embodiments, the membrane 340 may be omitted, or may include a material which does not substantially affect the operation of the membrane stack 300a. In still other embodiments, at least some compensation may be provided by a distinct rate-controlling membrane (not shown) in the membrane stack 300a.

In the illustrated embodiment, the membrane stack 300b also includes a rate-limiting membrane 350 positioned over the membrane 340. In the absence of a rate-limiting membrane 350, or another adjustment to the membrane stack 300b, a local concentration of dissolved oxygen within a bioprocess medium can be significantly less than the glucose concentration. This can result in the reactions discussed above being rate limited by the oxygen, and comparatively insensitive to changes in glucose concentration. The use of a rate limiting membrane 350 can overcome issues resulting from a comparative oxygen deficit by controlling the relative rates of glucose and oxygen diffusion therethrough. In some embodiments, the rate-limiting membrane 350 may comprise a silicone rubber material or another oxygen permeable membrane with small holes formed therein. These small holes or similar features allow a limited amount of glucose to pass through. The size, number, and positioning of these holes, along with the relative ratio of open area, can be used to control the relative glucose to oxygen concentration in the membrane. When suitably constrained, that the reactions discussed herein are no longer rate limited by the oxygen under certain conditions, to ensure that they are no longer substantially insensitive to changes in glucose concentration.

In addition to including two different membrane stacks 300a and 300b, where the membrane stack 300a is configured to provide a glucose-independent response to an O₂ concentration and the membrane stack 300b is configured to provide a glucose-dependent response to the that O₂ concentration, the sensor tip 100 includes an internal mask configured to allow the use of a single LED to measure the O₂ concentration at both of the O₂-sensitive membranes. The use of a single LED eliminates variance which may occur if comparative measurements were performed using a different LED to illuminate each membrane stack 300a and 300b.

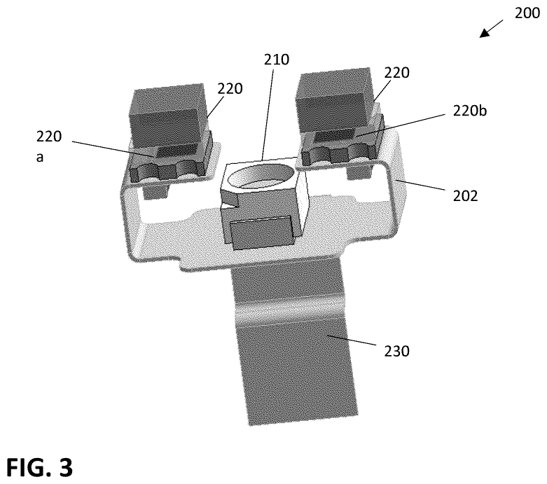

FIG. 3 is a perspective view of a front end electronics assembly configured for use with the dual membrane sensor tip 100 of FIG. 1A. The front end electronics assembly 200 includes a centrally supported LED 210 and a pair of photodiodes 220a and 220b supported on a frame 202. The frame includes or supports electrical connections between the photodiodes 220 and a flex circuit 230 which can be used to convey the output of the photodiodes 220, as well as to supply power to and control the LED 210. A filter 222 can overlie each of the photodiodes 220a and 220b. The filters 222 may be designed or selected to allow light of the wavelength emitted by the O₂-sensitive membranes in response to illumination by the LED 210, while filtering out other wavelengths of light, such as the wavelengths of light emitted by the LED 210. In some embodiments, the LED 210 may comprise a blue LED configured to emit light with a wavelength of around 450 nm, although other suitable LEDs or other light sources may also be used in other embodiments. The use of these filters 222 can ensure that the photodiodes 220a and 220b measure only the light which is emitted by the fluorescence of their respective membrane stack 300a and 300b in response to the illumination by the LED 210 and which is not quenched by the presence of dissolved oxygen.

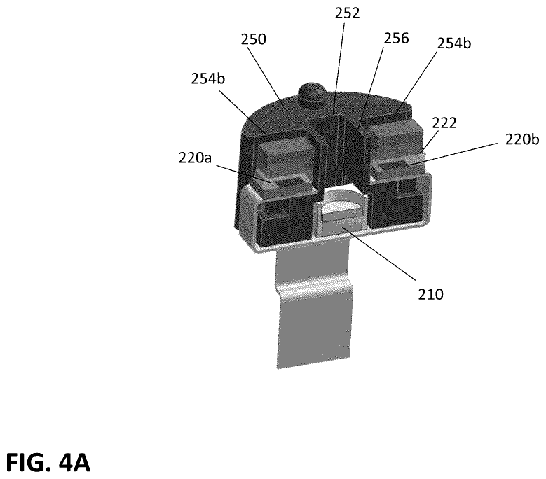

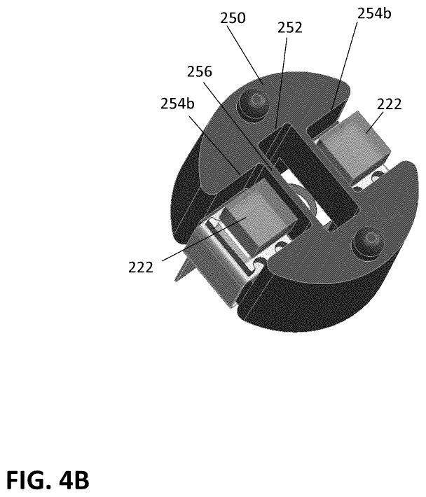

FIGS. 4A and 4B are perspective views of the front-end electronics assembly of FIG. 3, shown relative to an internal mask. As can be seen, the internal mask 250 is in the form of a opaque generally cylindrical plug having a plurality of apertures extending therethrough. A central aperture 252 is configured to overlie or surround the LED 210 and allow light emitted by the LED 210 to pass upward and through the central aperture 252. Outer apertures 254a and 254b allow light emitted by the membrane stacks 300a and 300b (not shown) to pass through the filters 220 and to the respective underlying photodiodes 220a and 220b. In the illustrated embodiment, these outer apertures 254a and 254b are in the form of notches which extend all the way to the outer sidewall of the internal mask 250, but in other embodiments a portion of the external mask may fully surround the outer apertures.

The outer apertures 254a and 254b are separated from the central aperture 252 by divider sections 256 of the internal mask 250 which shield the photodiodes 220 from direct illumination by the LED 210. In addition, the separating portions 256 of the internal mask 250 cooperate with other components of the sensor tip 100 to shield the photodiodes 220a and 220b from cross-illumination from the luminosity response of the opposing membrane stack, such that the photodiodes 220a and 220b responses provide an accurate measurement of the luminosity response of their adjacent membrane stack.

In the illustrated embodiment, the filters 222 overlying the photodiodes are supported by the front-end electronics assembly 200 although in other embodiments the filters 222 may be supported by or integrated into the internal mask 250. Any other suitable design of an internal mask can be used. For example, some embodiments of the internal mask may be formed from comparatively thin sections of an opaque material which define the central and outer apertures to constrain light flow as needed, while leaving other sections open.

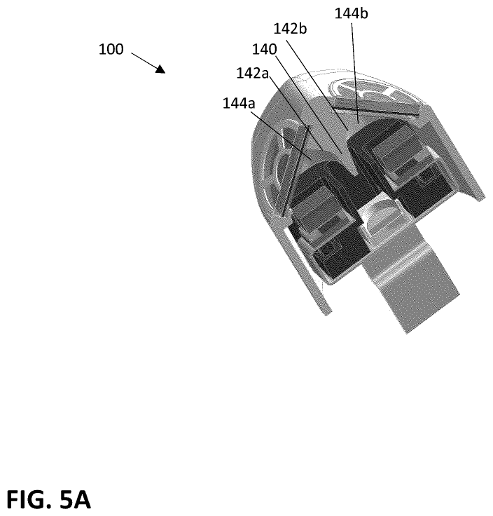

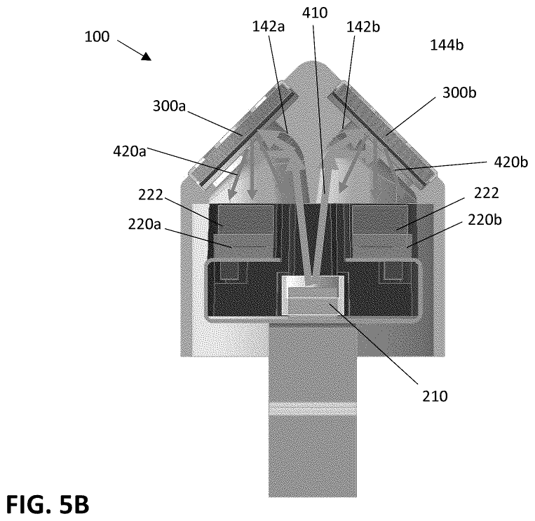

FIG. 5A is a perspective cross-sectional view of the assembled sensor tip 100. FIG. 5B is a side cross-sectional view illustrating the redirection of light within the sensor tip during operation. In particular, it can be seen that the interior of the sensor tip 100 includes a structural feature in the form of a downwardly extending wall 140 having first and second light redirection surfaces 142a and 142b on opposite sides. The wall 140 extends a sufficient distance towards the LED 210 and internal mask 250 to functionally separates the interior of the sensor tip 100 into two separate measurement chambers 144a and 144b.

As can be seen in FIG. 5B, light 410 is emitted from LED and passes upward and through the central aperture 252 (see FIG. 4A). Light 410 is then reflected by first and second light redirection surfaces 142a and 142b on opposite sides of downwardly extending wall 140 to direct light 410 towards membrane stacks 300a and 300b.

Membrane stack 300b, which can include a GOx-containing membrane 330, provides a fluorescence response in the form of emitted light 420b from the O₂-sensitive membrane 320. The intensity of the emitted light 420b is dependent on the glucose level in the process medium to which the sensor tip 100 and the membrane stack 300b is exposed, in addition to the O₂ levels in that process medium. Any direct light 410 which may have been reflected or scattered towards the filter 222 overlying photodiode 220b will be prevented from reaching the photodiode 220b due to the presence of the filter 222. In contrast, emitted light 420b will pass through the filter 222, and a magnitude of the current through the photodiode 220b will be indicative of the intensity of the emitted light 420b. The current can be converted, through appropriate circuitry, to an output signal indicative of the intensity of the emitted light 420b, such as an output voltage.

Similarly, membrane stack 300b, which can include a membrane 340 which does not contain GOx, provides a fluorescence response in the form of emitted light 420a from the O₂-sensitive membrane 320. The intensity of the emitted light 420a is independent of the glucose level to which the membrane stack 300b is exposed and is reflective only of the O₂ levels in the process medium to which the sensor tip 100 is exposed. Any direct light 410 which may have been reflected or scattered towards the filter 222 overlying photodiode 220b will be prevented from reaching the photodiode 220a due to the presence of the filter 222. In contrast, emitted light 420a will pass through the filter 222, and a magnitude of the current through the photodiode 220a will be indicative of the intensity of the emitted light 420a. The current can be converted, through appropriate circuitry, to an output signal indicative of the intensity of the emitted light 420a, such as an output voltage.

The combination of the membrane stack 300a and the photodiode 220a is equivalent to an integrated optical dissolved oxygen sensor, and the combination of the membrane stack 300b and the photodiode 220b is equivalent to an optical glucose oxidase sensor. A difference between the output signals from photodiodes 220a and 220b can therefore together be used to provide an indication of the glucose concentration in the measured process medium. This differential analysis can be performed in some embodiment by circuitry internal to the sensor, and such a sensor can provide as outputs measurements of glucose concentration and dissolved oxygen, as well as a temperature of the process medium as measured by the sensor. In addition to providing constant and/or periodic measurements of glucose concentration without the need for periodic grab measurements, such a sensor can also eliminate a need for a dedicated DO sensor and/or temperature sensor.

Additional processing of the received signals can also be performed to improve the accuracy of the glucose level measurement. Because many bioprocesses include the injection of oxygen-rich air, particularly in the later stages of a bioprocess when pure O₂ is often supplied to the process medium, the sensor tip 100 can be susceptible to measurement fluctuations resulting from the adherence of bubbles against or adjacent the membrane stacks 300a and 300b. While the sensor tip 100 includes sensing surfaces oriented at roughly 45 degrees to a longitudinal axis of the sensor tip, an optimal configuration for deflecting bubbles, some bubbles may nevertheless come into contact with the sensing surfaces and may remain there for a period of time. The O₂ concentrations in these bubbles will not be reflective of the overall O₂ levels in the process medium. Rather than being exposed to the same dissolved oxygen conditions at both membrane stacks 300a and 300b, adherence of a bubble to one of the membrane stacks 300a or 300b will expose the membrane stacks 300a and 300b to different dissolved oxygen conditions and will result in inaccuracies in determined glucose values using the output signals of photodiodes 220a and 220b. U.S. Pat. No. 11,137,354, the disclosure of which is hereby incorporated by reference in its entirety, describes examples of bubble filters which can be used to detect and/or compensate for the presence of bubbles adhering to a sensor surface.

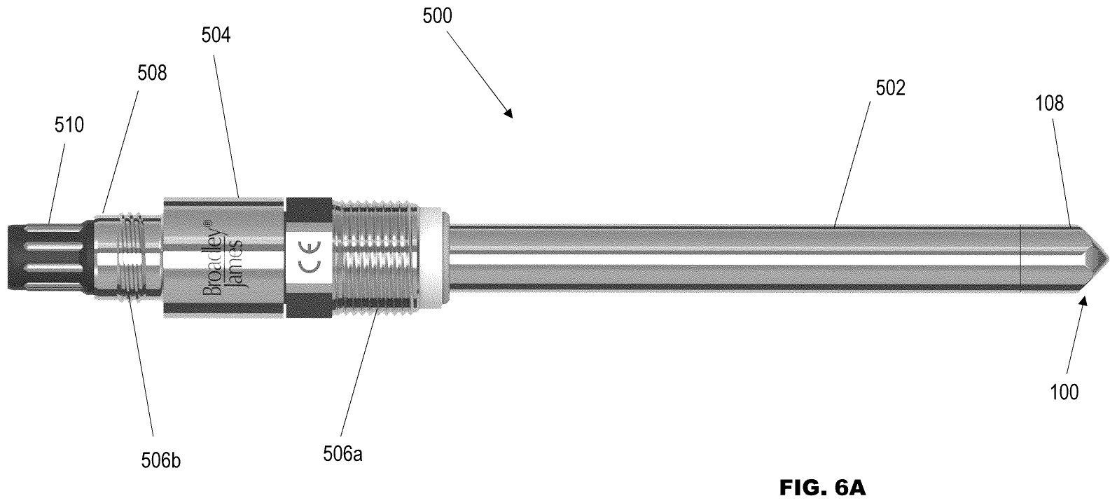

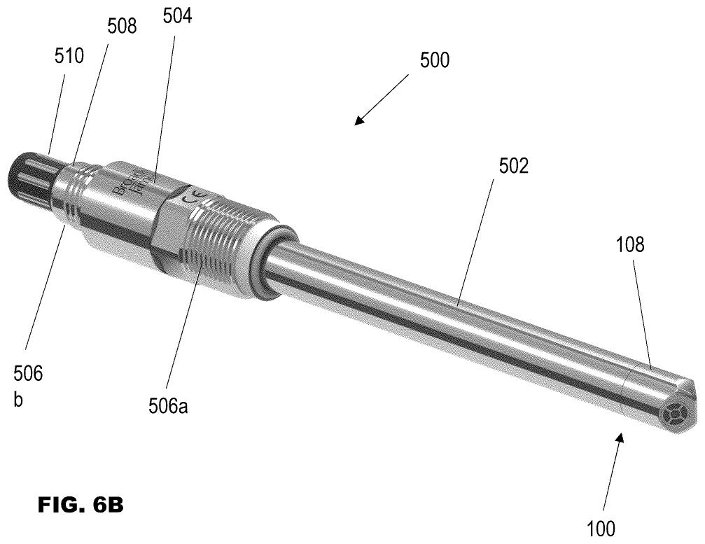

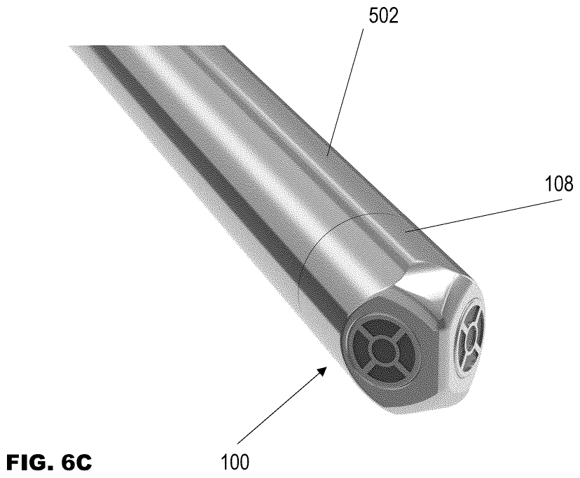

FIG. 6A is a top view illustrating a dual-membrane enzymatic sensor comprising the sensor tip of FIG. 1A. FIG. 6B is a perspective view illustrating the dual-membrane enzymatic sensor of FIG. 6A. FIG. 6C is another perspective view illustrating a distal portion of the dual-membrane enzymatic sensor of FIG. 6A.

The dual-membrane enzymatic sensor 500 includes a sensor tip 100 at its distal end. In some embodiments, the sensor tip 100 of dual-membrane enzymatic sensor 500 is similar to the sensor tip 100 of FIGS. 1A to 5B discussed above, although in other embodiments, other types and designs of enzymatic sensor tips may be utilized, as discussed in greater detail below.

Proximal of the sensor tip 100, the enzymatic sensor 500 includes a generally cylindrical distal portion 502 having an outer cross-sectional shape that is substantially similar to the outer cross-sectional shape of the generally cylindrical portion 108 of the sensor tip 100. In the illustrated embodiment, other than the pointed tip of the sensor tip 100, the distal section of the enzymatic sensor 500 has a substantially constant and smooth outer profile.

Proximal the generally cylindrical distal portion 502, the enzymatic sensor 500 includes a wider central portion 504 having an outer cross-sectional shape larger than the outer cross-sectional shape of the generally cylindrical distal portion 502. Although referred to as a wider central portion 504, the wider central portion 504 is not necessarily located in the exact center of the enzymatic sensor 500. Rather, in the illustrated embodiment, the generally cylindrical distal portion 502 is longer than the wider central portion 504 and the proximal portion 508 combined. Near the distal end of the wider central portion 504, the enzymatic sensor 500 includes a distal threaded portion 506a which, as discussed in greater detail below, can be used to secure the enzymatic sensor 500 relative to other components of a sensor assembly.

The enzymatic sensor 500 also includes a proximal portion 508 which includes a rear connector 510 which can be used to provide power to the enzymatic sensor 500 as well as communication between the enzymatic sensor 500 and an external system (not shown). The proximal portion 508 can also include a proximal threaded portion 506b which can be used to secure a cable or other connection relative to the rear connector 510 to provide a secure mechanical connection to the enzymatic sensor 500. In other embodiments, however, other mechanical connections may be provided, as an alternative to or in addition to threaded connections.

The dual-membrane enzymatic sensor 500 can also include internal components not specifically illustrated herein, including but not limited to circuitry and software to operate the optical sensors of the dual-membrane enzymatic sensor 500, and perform further processing on measurement signals. This circuitry and software can, for example, implement bubble filtering algorithms such as those disclosed in U.S. Pat. No. 11,137,354 to detect artifacts in measurement signals resulting from contact with bubbles within the process medium. These software algorithms or other processing can filter out spikes caused by these bubbles from the sensor signals. The enzymatic sensor may include a memory configured to store instructions for applying a filter to the measurement signals from some or all of the sensors of the enzymatic sensor 500, including measurement signals from each of the photodiodes. The application of the filter can generate a filtered output with reduced measurement inaccuracies due to the gas bubbles within the process medium as compared to the measurement signal. In some embodiments, the filter may determine a rate of change of a measurement signal and compare an absolute value rate of change to a threshold value and can in some particular embodiments output measurement signals only when the rate of change is less than a threshold value. Alternative and additional methods of filtering measurement signals which may be used are described in U.S. Pat. No. 11,137,354, and various embodiments of enzymatic sensors may implement some or all of these methods of filtering measurement signals and can also allow for user adjustment of such filtering. In some embodiments, some or all of the described filtering may be performed irrespective of the shape of the enzymatic sensor 500.

In some alternate embodiments, only a single enzymatic membrane may be included in the sensor tip. In some such embodiments, as depicted in other figures in the application, a single enzymatic membrane may be positioned orthogonal to a longitudinal axis of an enzymatic sensor to provide a substantially planar distal surface, although any other suitable arrangement and orientation of a single enzymatic membrane may also be used. In other alternate embodiments, more than two enzymatic membranes may be included in the sensor tip.

As discussed previously, the enzymes in enzymatic sensors such as glucose sensors and other enzymatic sensors such as lactate sensors are severely damaged by autoclaving or gamma sterilization methods. To provide a sterilized enzymatic sensor, alternative sterilization methods can be used, such as the use of Ethylene Oxide gas (ETO) to sterilize the components of the enzymatic sensor. However, the use of ETO can place constraints on the design of a sensor assembly, to ensure that the ETO is able to sterilize all necessary components of the sensor assembly and to allow timely removal of the ETO after the sterilization process. In addition, the conditions to which the sensor assembly can be exposed during the sterilization process, such as exposure to a vacuum, can place further constraints on the design of the sensor assembly to prevent damage to the sensor assembly.

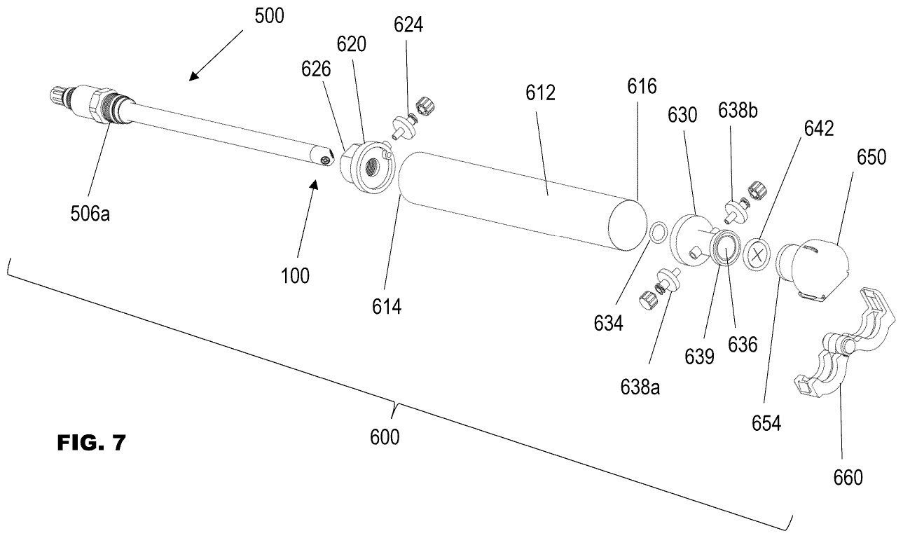

FIG. 7 is a perspective exploded view illustrating various components of one embodiment of a sterilizable enzymatic sensor insertion assembly comprising a dual-membrane enzymatic sensor such as the dual-membrane enzymatic sensor of FIG. 6A, comprising a sensor tip such as the sensor tip of FIG. 1A. The sterilizable enzymatic sensor assembly 600 includes the dual-membrane enzymatic sensor 500, as well as a number of components configured to be sequentially assembled prior to and following a sterilization procedure. The sterilizable enzymatic sensor assembly 600 includes a flexible tube 612 which may comprise a material such as TYVEK or any other suitable material. The material of the flexible tube 612 can be completely permeable to ETO or another suitable non-radiation sterilization agent. The flexible tube 610 can include at its proximal end 614 a proximal tube cap 620 having a proximal aperture extending therethrough. In addition, the proximal tube cap may include or be in fluid communication with an air vent 624 allowing air to exit therethrough when the flexible tube 612 is compressed or otherwise deformed to reduce an internal volume of the flexible tube 612. The air vent 624, along with all other ports and vents in the sensor assembly 600, is a filtered vent which includes a submicron filter to prevent microbial intrusion into the sterile interior of the completed sensor assembly 600.

The proximal tube cap 620 can also include a rear threaded portion 626 configured to engage the distal threaded portion 506a of the enzymatic sensor 500 to connect the enzymatic sensor 500 to the proximal tube cap 620 at a point in an assembly process.

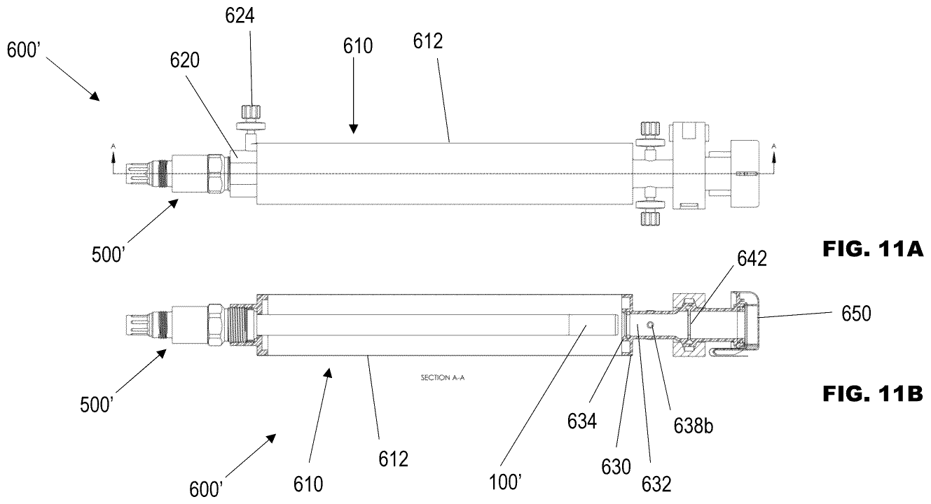

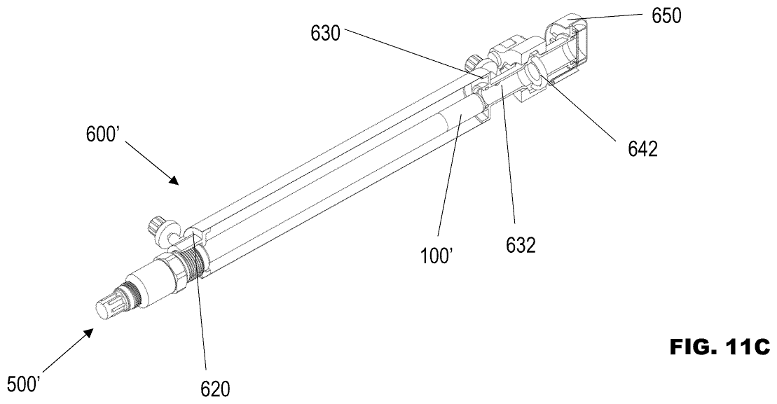

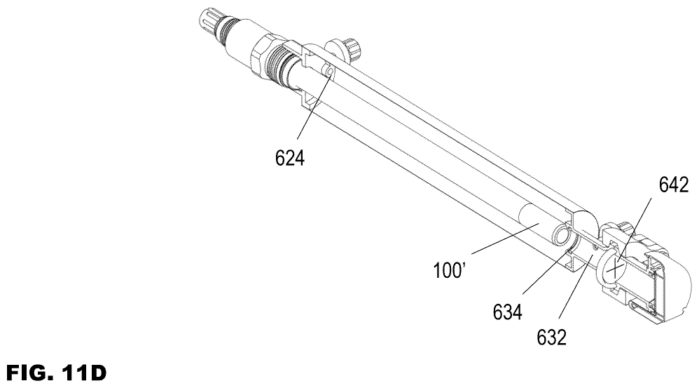

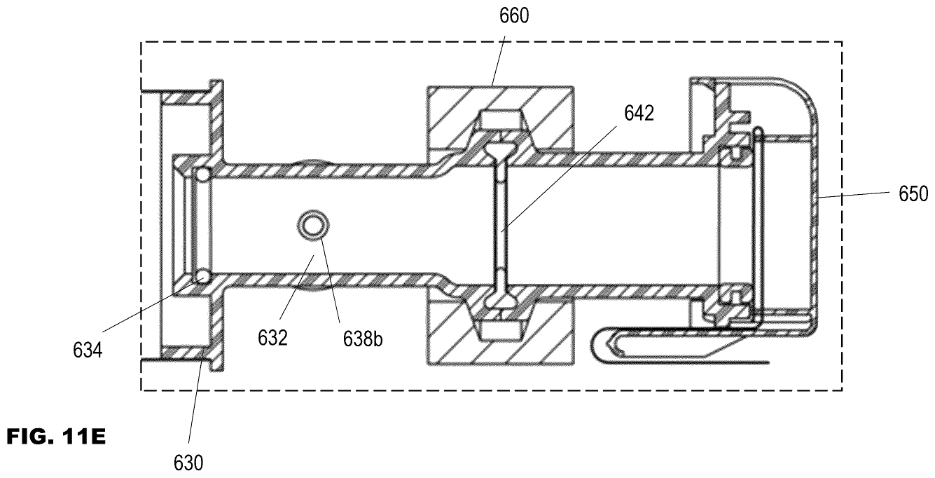

A distal tube cap 630 can be configured to be connected to the distal end 616 of the flexible tube 612. The distal tube cap 630 can comprise an internal storage chamber 632 (see, for example, FIG. 11B) having a gasket 634 such as an O-ring or other suitable scaling structure located at the proximal end of the internal storage chamber 632. An aperture 636 is located at the distal end of the internal storage chamber 632. A pair of access ports 638a and 638b are in fluid communication with the internal storage chamber 632 to allow the internal storage chamber 632 to be filled with a storage solution, as discussed in greater detail elsewhere herein. Like the air vent 624 discussed above, the access ports 638a and 638b are filtered access ports which include a submicron filter. A distal end of the distal tube cap 630 includes a radially extending flange 639 which can be used to secure the distal tube cap 630 to other components, as discussed in greater detail elsewhere herein.

A membrane 642 is dimensioned to occlude the aperture 636 at the distal end of the distal tube cap 630 when the membrane 642 is positioned adjacent the aperture 636. As discussed in greater detail elsewhere herein, the membrane 642 may be configured to allow the sensor tip 100 to push through the membrane 642 prior to use of the sensor assembly 600 in a measurement process. Prior to this, the membrane 642 may form a distal end of a storage chamber for retaining a storage fluid around the sensor tip 100, as discussed in greater detail elsewhere herein.

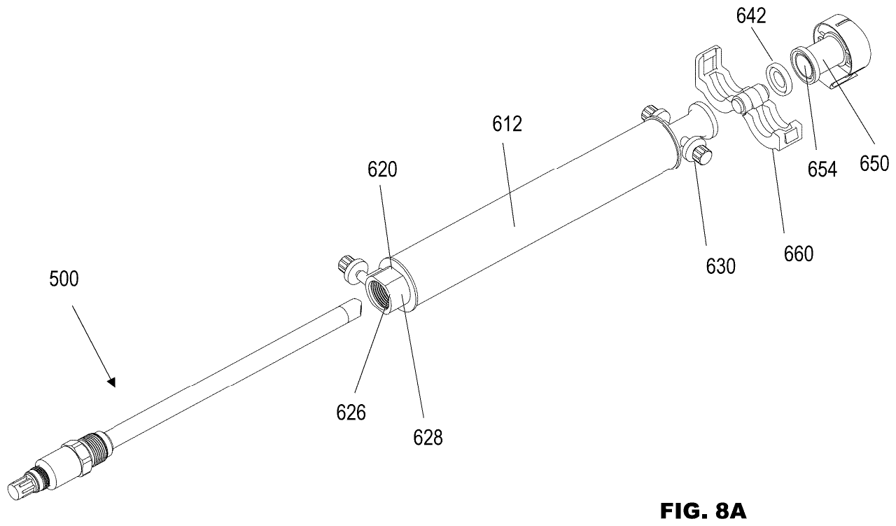

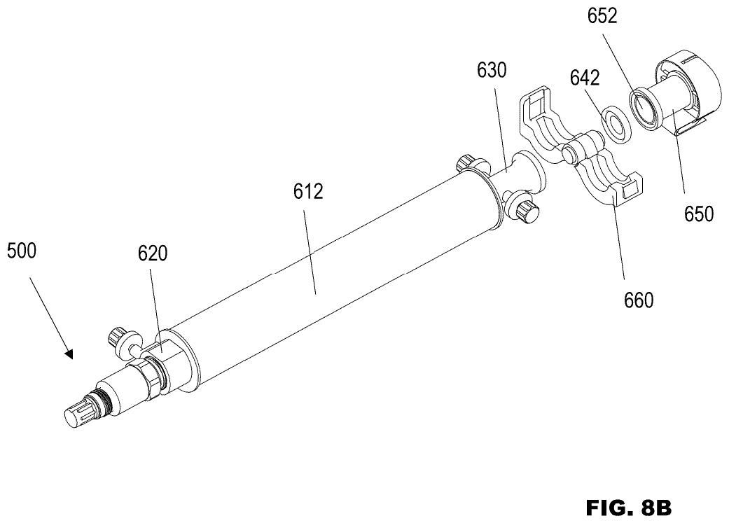

The sensor assembly 600 also includes an aseptic connector 650 having a radially extending flange 654 at the proximal end of the aseptic connector 650, surrounding a proximal aperture 652 (see FIG. 8A). As discussed in greater detail elsewhere herein, the aseptic connector 650 can allow an assembled and sterilized sensor assembly 600 to be connected to a bioprocess container while maintaining the sterility of the interior of the sensor assembly 600 and the bioprocess container. This sterile connection allows the sensor 500 to be inserted at least partially into the bioprocess container, exposing the sensor tip 100 to the interior of the bioprocess container without contaminating the bioprocess container.

The sensor assembly 600 also includes a clamp 660 dimensioned to be clamped over and retain the aseptic connector 650 to the distal tube cap 630, with the membrane 642 positioned therebetween to separate the internal storage chamber 632 of the distal tube cap 630 from the interior of the aseptic connector 650. In the illustrated embodiment, the clamp 660 comprises interior surfaces dimensioned to retain the radially extending flange 639 of the distal tube cap 630 and the radially extending flange 654 of the aseptic connector 650, preventing the distal tube cap 630 from being longitudinally translated away from the aseptic connector 650, so that the membrane 642 is held in place between the distal tube cap 630 and the aseptic connector 650.

The sensor assembly 600 can be partially assembled in a manner that leaves internal components of the sensor assembly 600 open and exposed to airflow. FIG. 8A is a perspective exploded view illustrating various components of the sterilizable enzymatic sensor assembly 600 of FIG. 7, in which a tube section of the insertion assembly is shown in an assembled state. In particular, the distal tube cap 630, the flexible tube 612, and the proximal tube cap 620 have been joined together to form a tube section 610 having a proximal aperture 622 extending through the proximal tube cap 620 and into the interior of the flexible tube 612 and a rear threaded portion 626 in the wider proximal section 628 of the proximal tube cap 620. Although it cannot be seen in FIG. 8A, it can be seen in other figures, such as FIG. 11B, that the gasket 634 is retained in place within the distal tube cap 630, such as within an interior groove formed or otherwise defined in an interior wall of the internal storage chamber 632, at or near a proximal end of the internal storage chamber 632.

Because the tube section 610 includes openings extending through both the proximal tube cap 620 and the distal tube cap 630, one of those two openings can be occluded without preventing airflow into or out of the interior of the tube section 610. In some embodiments, the sensor 500 may be inserted into the proximal end of the tube section 610 prior to a sterilization procedure, in order to reduce assembly required subsequent to the sterilization procedure to provide an assembled and sterilized sensor assembly.

FIG. 8B is a perspective exploded view illustrating the components of the sterilizable enzymatic sensor assembly as shown in FIG. 8A, with the sensor inserted into the distal section of the insertion assembly. As can be seen in other figures, such as FIG. 11B, the wider proximal section 628 of the proximal tube cap 620 is dimensioned to allow the distal threaded portion 506a of the sensor 500 to be inserted and engaged with the rear threaded portion 626 of the proximal tube cap 620, with the comparatively thinner generally cylindrical distal portion 502 of the enzymatic sensor 500 extending through the narrower proximal aperture 622.

The maximum length of the flexible tube 612, when fully extended, can be greater than a combined length of the generally cylindrical distal portion 502 and the sensor tip 100 of the enzymatic sensor 500. Because of this difference in length, the sensor 500 can be positioned within the tube section 610 such that the sensor tip 100 is located proximal of, and not in contact with, the gasket 634 or any other portion of the distal tube cap 630. This spacing permits airflow into the interior of the tube section 610 through the aperture 636 in the distal tube cap 630, reducing the potential for accidental occlusion of the aperture 636.

In some embodiments, an assembly process of the sensor assembly can proceed to a point where each component has a single open aperture extending into that component and permitting airflow into and out of the component, before a sterilization process is initiated. In other embodiments, however, a sterilization process may be initiated before an assembly process proceeds to such a point. By assembling the sensor assembly as much as possible before the sterilization process is initiated, however, the assembly steps required for final assembly can be reduced. In an embodiment such as those discussed herein, in which the final assembly steps can be performed within a discrete sterile package, reducing the complexity of the final assembly process can reduce a risk of damaging the sterile package during assembly, or other errors in the assembly.

FIG. 9 is a flow diagram depicting certain steps in a series of processes including sterilizing an enzymatic sensor assembly, preparing the sterilized enzymatic sensor assembly for storage, and inserting the sterilized enzymatic sensor assembly into a bioprocess container for use in measuring a property of a medium within the bioprocess container. Although illustrated as a single process, it will be understood that the various steps can and typically will be performed by different entities, and a significant amount of time may pass between the performance of certain steps. The overall process flow includes a number of discrete individual processes, and are only presented herein as a single process to illustrate an example of how these discrete processes can be performed on a given sensor assembly over time.

The process 700 includes a stage 705 at which a plurality of sensor components is provided. The plurality of sensor insertion assembly components can include discrete components of the structures illustrated in FIG. 8A, for example, or can include some or all of the individual pieces of those components prior to partial assembly to the point depicted in FIG. 8A. For example, in some embodiments, the individual components of tube section 610 may be provided and then assembled, while in other embodiments, an assembled tube section 610 may be provided.

The process 700 also includes a stage 710 at which pre-sterilization assembly is performed, where certain components are assembled in preparation for a sterilization process. In some embodiments, the pre-sterilization assembly comprises insertion of an enzymatic sensor into a tube section and securement of the enzymatic sensor to the tube section. In some embodiments, the enzymatic sensor may include a dual-membrane enzymatic sensor, while in other embodiments, the enzymatic sensor may include only a single enzymatic membrane. In some embodiments, the stage 510 may be omitted, and the separate components may be intended to be sterilized in their separated state.

The process 700 includes a stage 715 where a plurality of components of a partially assembled sensor assembly are packaged together in a sterile package. In particular, the sterile package includes a contiguous storage chamber, with each of the plurality of components of the partially assembled sensor assembly being packaged together in the same contiguous storage chamber. In some embodiments, at least a portion of the sterile package may be permeable to a sterilization agent such as ETO. In some embodiments, a cross-sectional area of the package which is permeable to the sterilization agent may be greater in size than a combined footprint of the plurality of components. In some embodiments, the package may comprise a sheet of material permeable to a sterilization agent which is bonded or otherwise adhered to a section of transparent and flexible material, which is not permeable to the sterilization agent.

In some embodiments, additional preparation steps may be performed in order to ensure that all exposed surfaces of the components of the sensor assembly can be sterilized, and that exposure to vacuum conditions during the sterilization process will not damage the components of the sensor assembly. For example, in some embodiments, caps on the various ports and vents within the components of the sensor assembly may be loosened or removed prior to packaging the components in the sterile package. Doing so can prevent pressure differentials from forming across the filters when the package is exposed to vacuum conditions. A sealed filter cap or plug on one side of the filter can result in a potentially damaging pressure differential. In addition, a loose or removed cap can facilitate better circulation of a sterilization agent such as ETO through the filter material, as well as the subsequent removal of that sterilization agent at a later point in the sterilization process.

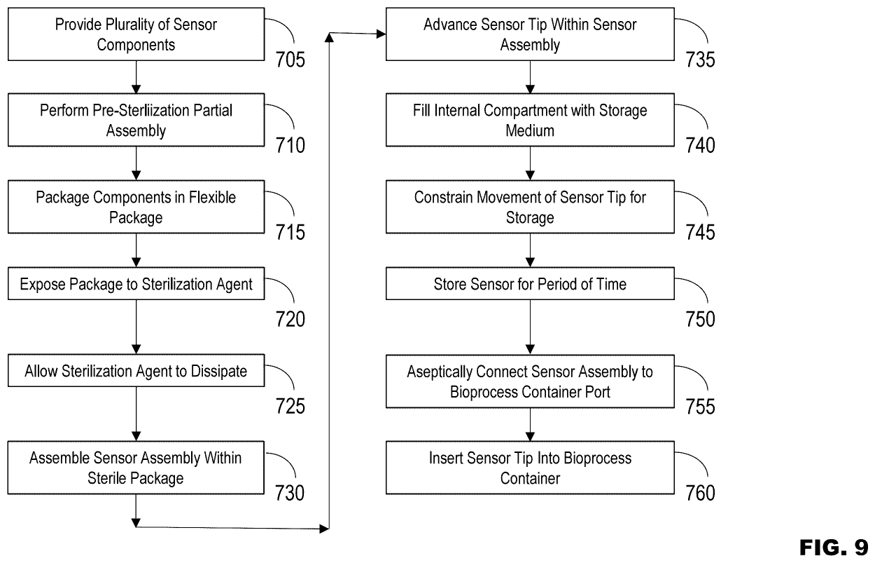

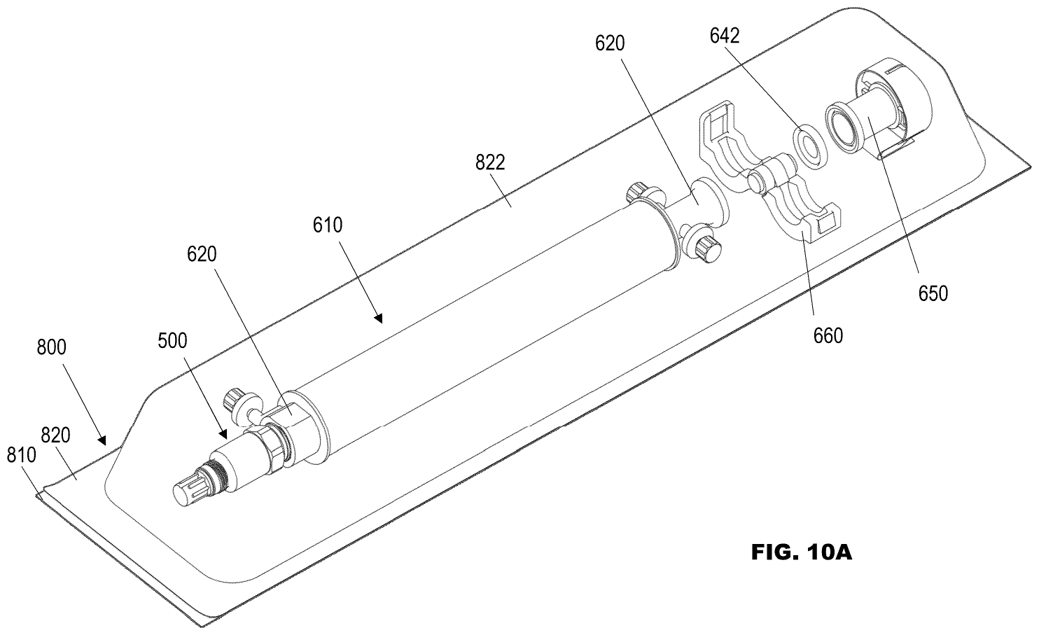

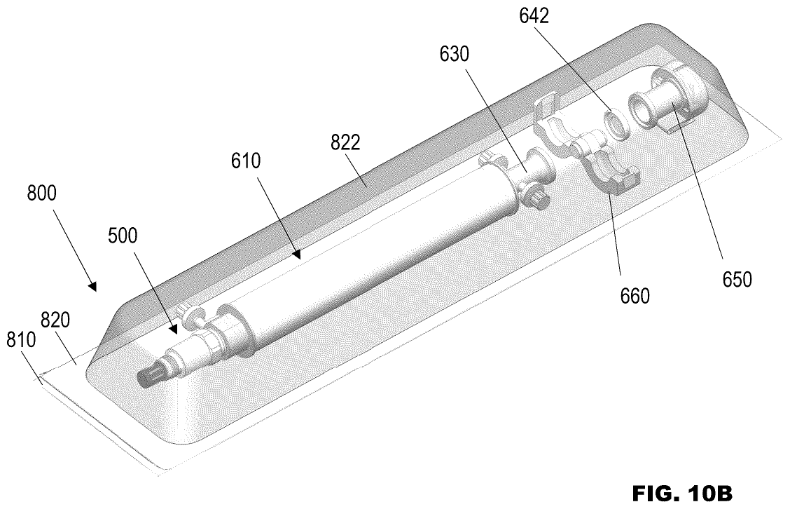

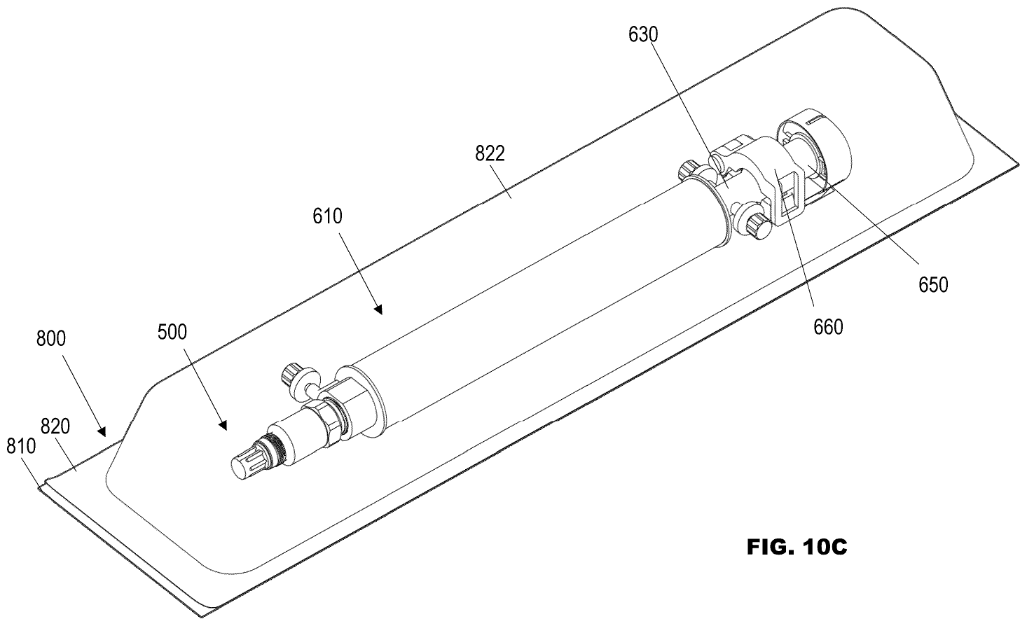

FIG. 10A is a perspective view of the partially assembled sensor assembly, disposed inside a sterilization package which includes a material permeable to a sterilization chemical. FIG. 10B is an alternative illustration of the perspective view of the partially assembled sensor assembly of FIG. 10A, with shading to illustrate certain features of the partially assembled sensor assembly. The package 800 comprises a generally planar layer 810 of material which is adhered to another layer 820 including a raised central portion 822 dimensioned to provide sufficient clearance for the components of the partially assembled sensor assembly.

In some embodiments, the generally planar layer 810 of material can include a flexible material which is permeable to the desired sterilization agent. In some particular embodiments, the generally planar layer 810 can comprise a layer formed from polyethelene fibers, such as a layer of TYVEK material, which is permeable to ETO. In some embodiments, the other layer 820 including the raised central portion 822 may be formed from a layer of transparent plastic which need not be permeable to the desired sterilization agent. Because both the layers 810 and 820 of the package 800 are flexible, the contents of the package 800 can readily be manipulated through the walls of the package 800 and the contents of the package 800 are visible during this process.

In some embodiments, the components of the partially assembled sensor insertion assembly can be provided in a position relative to one another which generally corresponds to their ultimate position in the assembled sensor assembly. In some embodiments, support structures or other supplemental components which need not form a part of the finished sensor assembly can also be included within the sterile, flexible package, to assist in the assembly of the sensor insertion assembly. In other embodiments, the package can include only the components which will ultimately form the assembled sensor insertion assembly, and the materials and dimensions of the package can be selected to provide sufficient flexibility to readily assemble the sensor insertion assembly within the package without breaching the sterility of the package.

In the embodiment shown in FIG. 10A, the tube section 610, with the sensor 500 inserted through the proximal tube cap 620, is located at one side of the package 800. The aseptic connector 650 is located at the other side of the package 800 with the clamp 660 positioned between the aseptic connector 650 and the distal tube cap 630 of the tube section 610. The membrane 642 is also located between the aseptic connector 650 and the distal tube cap 630 of the tube section 610.

Returning to FIG. 9, the process 700 includes a stage 720 where the sterile package such as the sterile package 800 of FIG. 10A is exposed to the sterilization agent, allowing the sterilization agent to pass through a portion of the sterile package and expose both the exterior and interior of the plurality of components of the sensor assembly to the sterilization agent. As noted above, the sterilization agent may comprise ETO.

Exposure to a sterilization agent such as ETO will sterilize the surfaces with which the sterilization comes into contact. The exposure to a sterilization agent during the sterilization process must therefore include exposure of all the exposed surfaces of the of components within the sterilization package, both outside of and within the various components of the sensor assembly. While some internal parts of the sensor assembly, such as the interior of an enzymatic optical sensor, may remain hermetically sealed and thus not exposed to the sterilization operation, such parts of the sensor assembly will remain hermetically sealed during operation, and need not be sterilized by exposure to the sterilization agent.

Exposure of the sterile package to the sterilization agent may include controlling certain additional conditions to which the sterile package is exposed, including but not limited to the humidity to which the sterile package is exposed. The exposure conditions, and length of exposure of the sterile package, to the sterilization agent can be controlled to ensure that the plurality of components of the sensor assembly undergo sufficient exposure to the sterilization agent to fully sterilize the components. In some embodiments, the exposure to a sterilization agent such as ETO may last for several hours.

The process 700 also includes a stage 725 where the sterilization agent is allowed to dissipate from the sterile package, so that none of the sterilization agent remains within the sterile package or within any of the components of the sensor assembly. In some embodiments, this may include exposure of the sterile package to reduced pressure or to vacuum. Other than hermetically sealed sections, such as the interior of the sensor tip, the remainder of the components of the sensor assembly remain open to airflow, to allow the sterilization agent to be fully removed in a timely fashion. The open components allow the pressure within and around the components to remain equalized, preventing the creation of pressure differentials within the sensor components either during the sterilization process or during the removal of the sterilization agent, which could cause damage to or otherwise impact the components of the sensor assembly.

Subsequent to the removal of the sterilization agent in stage 725, the components of the sensor assembly have been sterilized with ETO or another suitable sterilization agent, which has now been removed. A partially assembled sensor insertion assembly can be provided, packaged within a sterile and flexible package in a manner which permits the components of the partially assembled sensor insertion assembly to be moved relative to one another and mechanically manipulated.

In some embodiments, additional steps may be included, such as transport steps. For example, prior to the sterilization process and removal of the sterilization agent, a packaged and partially assembled sensor assembly can be delivered to a dedicated sterilization facility equipped to perform an ETO sterilization process or similar sterilization process. After removal of the sterilization agent, the now-sterilized, packaged, and partially assembled sensor assembly can be transported to another facility for further assembly and other steps discussed below.

The process 700 also includes a stage 730 at which the sterilization process has been completed, and further assembly steps are performed to place the sensor assembly in a state where the interior of the sensor assembly is sealed from direct unfiltered airflow or other exposure. In some embodiments, this can be preceded by removal of the components of the sensor assembly from the sterilization package which, along with subsequent steps including but not limited to those discussed in stages 735 and 740, may be performed in a clean hood or other sterile environment. In a particular embodiment, the distal tube cap and the aseptic connector are brought together, with the membrane held between the distal tube cap and the aseptic connector, and the clamp sealed around the flanges at the facing ends of the distal tube cap and the aseptic connector. This assembly process can form a first internal chamber extending through the tube section and into the internal storage chamber of the distal tube cap, and a second internal chamber within the aseptic connector and separated from the first internal chamber by the membrane held in place between the distal tube cap and the aseptic connector.

FIG. 10C is a perspective view of the sensor assembly of FIG. 10A, shown in an assembled state within the sterilization package. As can be seen in FIG. 10A, the distal tube cap 630 and the aseptic connector 650 have been brought together, with the membrane 642 is held in place therebetween (not visible in FIG. 10C, but visible in FIG. 11B, for example). The clamp 660 has been closed, to enclose at least the facing radially extending flange 639 of the distal tube cap 630 and the radially extending flange 654 of the aseptic connector 650, preventing translation of the tube section 610 away from the aseptic connector 650 and sealing the interior chambers within the sensor assembly 600 from nonsterile airflow into the interior chambers of the sensor assembly 600.

FIG. 11A is a top plan view of a sensor assembly similar to the sensor assembly of FIG. 10A in an assembled state. FIG. 11B is a side partial cross-sectional view of the assembled sensor assembly, in which the sensor assembly is shown in a cross-section along the section line A-A of FIG. 11A to display the sensor disposed within the sensor assembly. FIG. 11C is a perspective partial cross-sectional view of the assembled sensor assembly of FIG. 11B. FIG. 11D is another perspective partial cross-sectional view of the assembled sensor assembly of FIG. 11B. FIG. 11E is a detail view of a portion of the sensor assembly of FIG. 11B including the distal sensor cap and the aseptic connector.

The sensor assembly 600′ of FIG. 11A is similar to the sensor assembly 600 of FIG. 10A, but differs in that the design of the sensor 500′ is slightly different than the design of the sensor 500 of the sensor assembly 600. In particular, the sensor 500′ includes a sensor tip 100′ with a generally planar face. The sensor 500′ can be, for example, an enzymatic sensor with only a single enzymatic membrane, or an enzymatic sensor in which multiple sensing membranes are disposed on a single planar face of the sensor tip 100′. The sensor assembly 600′ is otherwise similar to the sensor assembly 600. The alternative sensor 500′ is depicted to illustrate that the sensor assembly design and packaging methods can be used with alternative sensor designs, including sensor designs and types other than the design of sensor 500 having a dual-membrane sensor tip 100.

It can be seen in FIG. 11B that the sensor tip 100′ is spaced apart from the gasket 634 at the proximal end of the internal storage chamber 632 in the distal tube cap 630, and that the flexible tube 612 is sufficiently extended to provide this clearance. As can best be seen in FIG. 11D, the internal chamber within the flexible tube 612 surrounding the inserted section of the sensor 500′ is in fluid communication with the filtered air vent 624, allowing air to escape through the air vent 624 as the volume of the flexible tube 612 is reduced. It can also be seen in FIGS. 11B, 11C, and 11D, that the filtered access port 638b is in fluid communication with the internal storage chamber 632 in the distal tube cap 630 at a location between the gasket 634 at the proximal end of the internal storage chamber 632 and the membrane 642 scaling the distal end of the internal storage chamber 632.

In the illustrated embodiment, the membrane 642 comprises a thicker outer ring seated within radially symmetric grooves in the facing surfaces of the distal tube cap 630 and the aseptic connector 650, so that the portions of the distal tube cap 630 and the aseptic connector 650 radially outward of the membrane 634 can be brought into contact with one another, and a thinner portion of the membrane 634 radially inward of the thicker outer ring is held between portions of the distal tube cap 630 and the aseptic connector 650. The generally matching shapes of the membrane 634 and these radially symmetric grooves in the distal tube cap 630 and the aseptic connector 650 facilitate proper placement of the membrane 634 during the assembly process, when the distal tube cap 630 and the aseptic connector 650 are brought together on either side of the membrane 634 and clamped together using the clamp 660. As can be seen in FIG. 11D, the membrane 634 may be scored or otherwise selectively weakened to facilitate eventual rupture of the membrane 634 if the sensor tip 100′ is advanced through the membrane 634.

Referring again to FIG. 9, the process 700 can then move to a stage 735 where the distal tube cap can be moved in a proximal direction to insert the sensor tip through the gasket within the distal tube cap. Because the internal diameter of the gasket can be slightly less than the outer diameter of the sensor tip and the immediately proximal portion of the sensor, the contact between the gasket and the outer surface of the sensor can close the proximal end of the internal storage chamber within the distal tube cap, defining a sealed internal region of the sensor assembly which contains the enzymatic membrane or membranes of the sensor tip and is in fluid communication with the sterile access ports of the distal tube cap. In some embodiments, the sterilized sensor assembly may be removed from the sterilization package prior to performing these steps, while in other embodiments, the assembly may be removed from the sterilization package at a different point in the process.

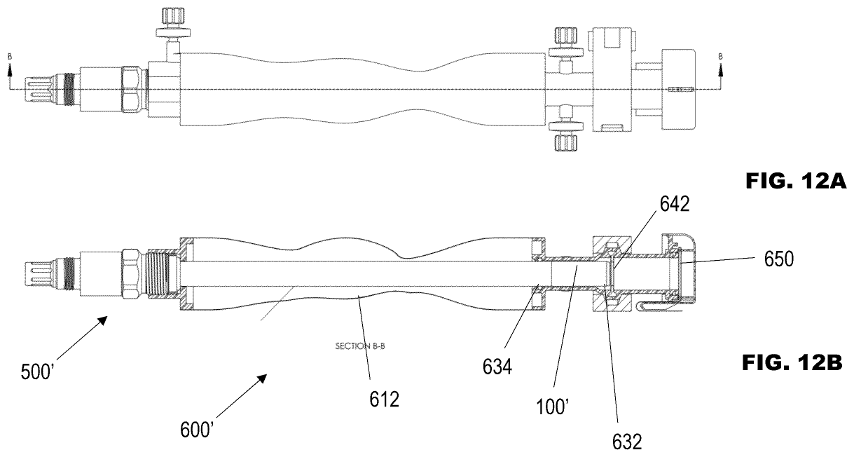

FIG. 12A is a side view of the sensor assembly of FIG. 11A, in which the sensor has been advanced to a distal position in preparation for storage. FIG. 12B is a side partial cross-sectional view of the sensor assembly of FIG. 12A, in which the sensor assembly is shown in a cross-section along the section line B-B of FIG. 12A to display the sensor disposed within the sensor assembly.

It can be seen in FIG. 12A that the advancement of the sensor tip 100′ into the internal storage chamber 632 within the distal tube cap 630 has partially collapse the flexible tube 612, reducing the distance between the proximal tube cap 620 and the distal tube cap 630. The compression of the internal volume of the flexible tube 612 can be compensated for by the release of air through filtered air vent 624. The sensor tip 100′ can be advanced to a position where the distal end of the sensor tip is in contact with or adjacent the membrane 642 without piercing or rupturing the membrane 642.

Although illustrated in a position immediately adjacent to the membrane 642, a range of positions of the sensor tip 100′ can result in the formation of a sealed internal storage chamber 632 within the distal tube cap 630, as long as the sensor 500′ has been advances sufficiently far that the portion of the sensor 500′ in contact with the gasket 634 is of sufficient cross-sectional dimension to contact the entire interior edge of gasket 634.

Referring again to FIG. 9, the process 700 can include a stage 740 where the internal storage chamber of the distal tube cap is filled in a sterile manner with a storage solution using the filtered access ports of the distal tube cap. This allows the enzymatic membrane to be kept wet during storage, enabling long-term storage of the sensor assembly without degradation of the enzymatic membrane. With this wet storage of the sensor assembly, the sensor assembly can be stored for an extended period of time. Subsequent to filling the internal storage chamber, the filtered ports can have their covers replaced and can in some embodiments be permanently sealed. In some embodiments, the sensor assembly can be stored for months or longer before being used. In a particular embodiment, the sensor assembly can be stored for 18 months or more before being used.

The process 700 can also include a stage 745 where movement of the distal tube cap relative to the proximal tube cap is constrained. In order to ensure that the sensor tip remains within the internal storage chamber, without being withdrawn from the internal storage chamber and releasing the storage material, or being further inserted through the membrane to rupture the membrane during a storage period, movement of the sensor relative to the remainder of the sensor assembly may be constrained. In one embodiment, a clip having a first portion configured to be connected to the distal tube cap and a second portion configured to be connected to the proximal tube cap may be used to maintain a desired spacing between the distal tube cap and the proximal tube cap. Because the sensor is rigidly secured relative to the proximal tube cap, maintaining a specific spacing between the distal tube cap and the proximal tube cap directly commands a specific position of the sensor tip relative to the internal storage chamber in the distal tube cap. In some embodiments, movement may be constrained before the internal storage chamber is filled with the storage solution.

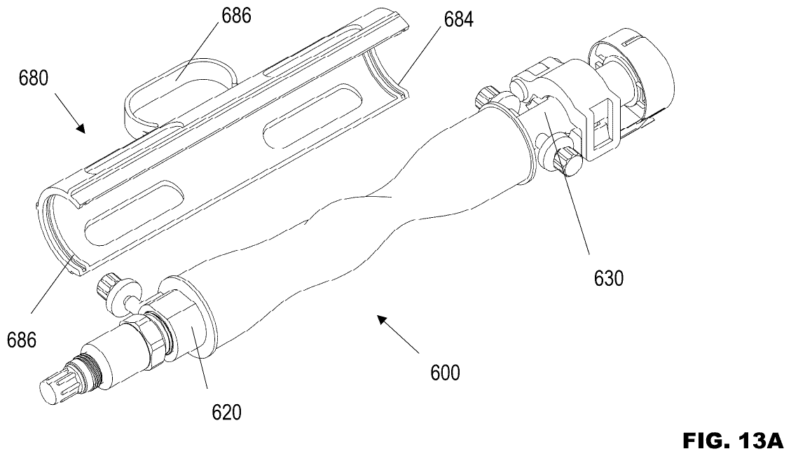

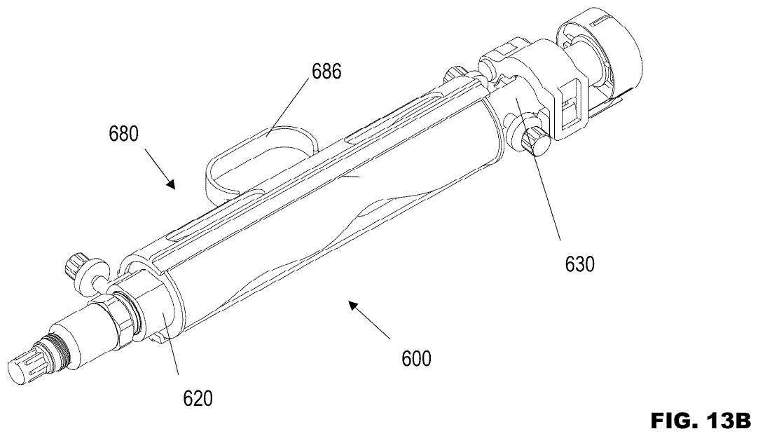

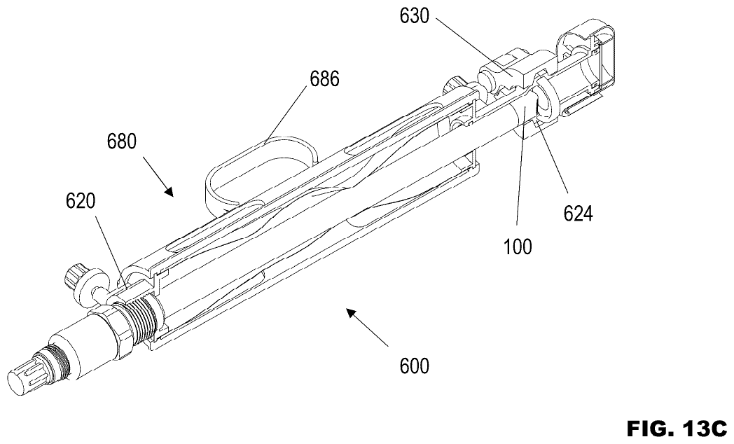

FIG. 13A is a perspective view of a sensor assembly such as the sensor assembly of FIG. 12A, in which a sensor has been advanced so that a sensor tip is positioned within the internal storage chamber within the distal tube cap. In the embodiment illustrated in FIG. 13A, the sensor assembly 600 includes a sensor 500 having a sensor tip 100 with two enzymatic membranes, such as the sensor of FIG. 6A. The sensor assembly 600 has been moved into a storage configuration, in which the sensor tip 100 is positioned within the sealed internal storage chamber 632 within the distal tube cap 630, with the proximal end of the storage chamber 632 being sealed by the fit between a portion of the sensor 500 and the gasket 634.

A retaining clip 680 is disposed adjacent the tube section 610 of the sensor assembly 600. The retaining clip 680 has a generally U-shaped cross-section and includes a handle 686 which can be used to hold the clip 680. A proximal groove 682 is dimensioned to receive a radially extending flange of the proximal tube cap 620, and a distal groove 684 is dimensioned to receive a radially extending flange of the distal tube cap 630.

When the retaining clip 680 is clipped onto the tube section 610 of the sensor assembly, as illustrated in FIG. 13B, movement of the proximal tube cap 620 relative to the distal tube cap 630 is inhibited. As can be seen in FIG. 13C, the spacing between the proximal groove 682 and the distal groove 684 of the retaining clip 680 is dimensioned to command a specific position of the sensor tip 100 within the internal storage chamber 632 of the sensor assembly 600. The retaining clip can be used to retain the sensor tip 100 in the desired location during storage, and can be left in place during installation of the sensor assembly relative to a port of a bioprocess container and removed when the sensor is intended to be inserted into the bioprocess container.

Referring again to FIG. 9, the process 700 can also include a stage 750 where the sensor is stored for a desired period of time, with the storage fluid in the internal storage chamber preventing the enzymatic membrane from drying out and degrading.

Referring still to FIG. 9, the process 700 can then include a stage 755 where the aseptic connector of the sensor assembly is connected to an aseptic connector of a port in a wall of a bioprocess container. This aseptic connection provides a sterile connection between the sensor assembly and the port.

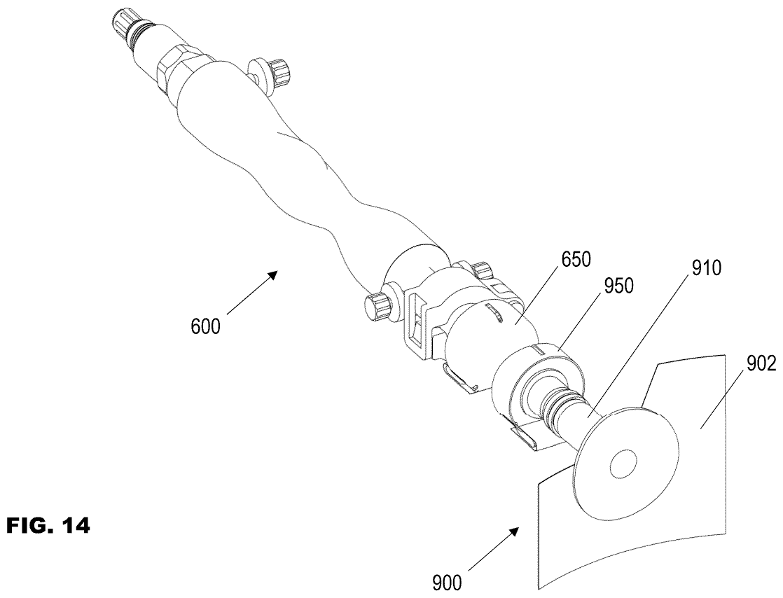

FIG. 14 is a perspective view of the sensor assembly of FIG. 13A, shown being connected to a port in a wall of a bioprocess container. A connection will be formed between the aseptic connector 650 of the sensor assembly 600 and a compatible aseptic connector 950 in a port 910 in the wall 902 of a bioprocess container 900. At the time the connection is made, the membrane 642 internally separating the distal tube cap 630 from the aseptic connector 650, preventing the components of the aseptic connector from being exposed to the storage solution while the aseptic connection with the port 910 of the bioprocess container 900 is made.

Referring again to FIG. 9, the process 700 can also include a stage 760 where the sensor is advanced through the aseptic connection and the port and into the interior of the bioprocess container. If not already done, this process can include the removal of a storage clip to allow movement of the sensor. The process can include rupturing the membrane at the distal end of the internal storage chamber of the sensor assembly, but the remainder of the membrane may cooperate with the inserted sensor to inhibit or prevent the storage solution from flowing into the bioprocess container. In addition, because the dimensions of the internal storage chamber are similar to the dimensions of the sensor tip, a comparatively small volume of storage solution is required to fill the chamber and maintain wet storage of the enzymatic membrane or membranes, reducing the possible impact on the bioprocess container contents if some or all of the storage solution were to flow into the bioprocess container.

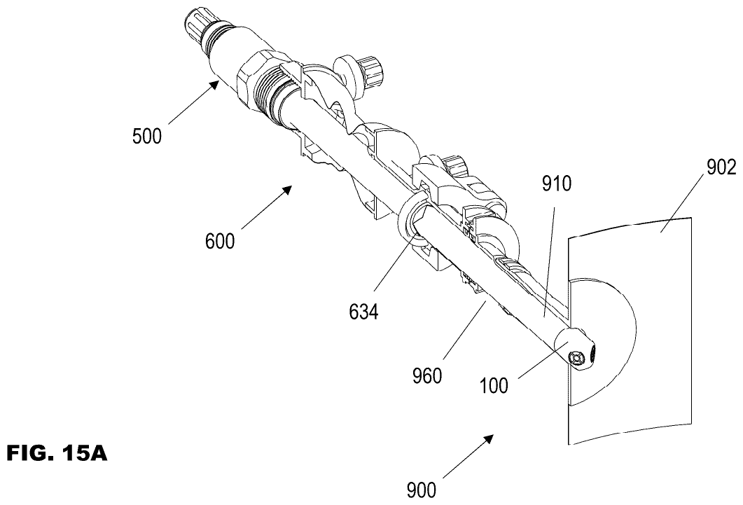

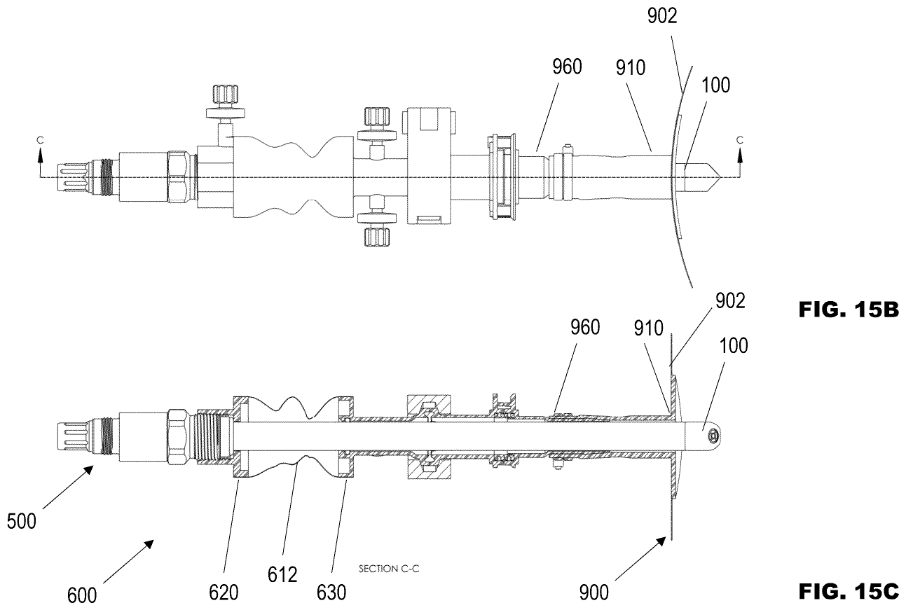

FIG. 15A is a perspective view of the sensor assembly of FIG. 14, with the sensor having been advanced to a measurement position in which the sensor tip of the sensor extends into a bioprocess container, shown in partial cross-section. FIG. 15B is a top plan view of the sensor assembly of FIG. 15A. FIG. 15C is a side partial cross-sectional view of the sensor assembly of FIG. 15A, in which the sensor assembly is shown in a cross-section along the section line C-C of FIG. 15B to display the sensor disposed within the sensor assembly.

In the illustrated embodiment, the sensor 500 has been advanced through the aseptic connection 960 formed between the sensor assembly 600 and the port 910, to a point at which the sensor tip 100 is fully within the bioprocess container 900. The travel range of the sensor tip 100 is constrained at least by the spacing between the facing surfaces of the distal tube cap 630 and the proximal tube cap 620. In some embodiments, the sensor 500 can be advanced further than illustrated in FIG. 15C, with the flexible tube 612 further collapsing. The filtered air vent 624 can allow air to pass therethrough as the internal volume of the flexible tube 612 decreases.

Referring again to FIG. 9, the process 700 can also include a stage 765 where the glucose concentration of a bioprocess medium within the bioprocess container is monitored. In contrast to daily pulls or other periodic pulls to sample the bioprocess medium at wide intervals and make occasional glucose measurements, the enzymatic sensor of the sensor assembly can remain inserted into the bioprocess container for an extended period of time, even for the entire duration of a bioprocess, and can continually monitor the glucose concentration.

The glucose measurements—along with other measurements which can be provided by the sensor, such as dissolved oxygen concentration, temperature, and any other measurements which can be provided by components integrated into the sensor—can be used in a control system for maintaining a glucose concentration at desired concentrations over the lifetime of the bioprocess. Because changes in glucose concentration can be quickly detected due to the online monitoring of the glucose concentration, the scope of corrective adjustments can be constrained, minimizing overcorrection and allowing the glucose concentration to be maintained within a desired range over the lifetime of the bioprocess.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

In particular, while the embodiments above have been discussed in the context of glucose sensing, the described embodiments can be used in conjunction with other enzymatic sensors. In addition, other configurations of enzymatic sensors not specifically provided are also within the scope of the present application. For example, some embodiments of enzymatic sensor may have redundant sensing elements to detect and compensate for bubble impacts or other measurement inconsistencies, such that if an anomalous measurement signal is detected, an alternate measurement signal from a redundant sensing element may be used in place of the anomalous measurement signal. In some embodiments, different types of enzymatic sensing elements may be combined into a single enzymatic sensor body, such as to provide an indication of both glucose concentration and lactate concentration, or any other parameter which can be detected or measured using an enzymatic sensor. The ETO sterilization processes described herein may be used to sterilize non-enzymatic sensors and may be used to sterilize any structure which can be degraded or damaged by exposure to autoclaving conditions and gamma radiation, but which will not be significantly affected by exposure to ETO. In particular, such structures may be sterilized in a partially assembled form within an ETO-permeable package, and then further assembled within the package.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present.

1) An enzymatic sensor, comprising:
a internal chamber;
a first window in a wall of the internal chamber;
a glucose-reactive membrane stack positioned over the first window, the glucose-reactive membrane stack at least partially exposed to an exterior of the enzymatic sensor;
a second window in a wall of the internal chamber;
a glucose-insensitive membrane stack positioned over the second window, the glucose-insensitive membrane stack at least partially exposed to the exterior of the enzymatic sensor;
a light source disposed within the internal chamber and configured to illuminate the glucose-reactive membrane stack through the first window and to illuminate the glucose-insensitive membrane stack through the second window;
a first sensing photodiode optically shielded from the second window and configured to detect a first fluorescence response of the glucose-reactive membrane stack to illumination from the light source; and
a second sensing photodiode optically shielded from the first window and configured to detect a second fluorescence response of the glucose-insensitive membrane stack to illumination from the light source.

2) The sensor of claim 1, wherein the first window is formed in a first face of the sensor and the second window is formed in a second face of the sensor, the first face oriented at an angle to the second face.

3) The sensor of claim 1, wherein the glucose-reactive membrane stack and the first sensing photodiode form a part of a glucose-sensing component configured to provide an indication of glucose levels in a process medium to which the sensor is exposed.

4) The sensor of claim 1, wherein the glucose-insensitive membrane stack and the second sensing photodiode form a part of a dissolved oxygen sensing component configured to provide an indication of dissolved oxygen levels in a process medium to which the sensor is exposed.

5) The sensor of claim 1, additionally comprising a processor configured to provide an output signal indicative of glucose concentration in a process medium to which the sensor is exposed.

6) The sensor of claim 5, wherein the processor is configured to determine the glucose concentration based at least in part on a difference between an indication of oxygen concentration at the glucose-reactive membrane stack and an indication of oxygen concentration at the glucose-insensitive membrane stack.

7) The sensor of claim 6, wherein the processor is further configured to determine the glucose concentration based at least in part on a measured temperature of the process medium to which the sensor is exposed.

8) The sensor of any of claim 5, wherein the processor is further configured to determine the glucose concentration based at least in part on an absolute measurement of the dissolved oxygen levels in the process medium to which the sensor is exposed.

9) The sensor of claim 1, wherein the glucose-reactive membrane stack comprises glucose oxidase.

10) The sensor of claim 9, additionally comprising a rate-limiting membrane disposed on an outer surface of the glucose-reactive membrane stack.

11) The sensor of claim 10, wherein the rate-limiting membrane comprises an oxygen-permeable material comprising a plurality of holes extending therethrough to control glucose diffusion through the rate-limiting membrane.

12) The sensor of claim 1, wherein each of the first and second sensing photodiodes are shielded by a filter which permits the fluorescent responses to pass therethrough which filtering any illumination from the light source.

13) The sensor of claim 1, wherein the glucose-reactive membrane stack comprises a first dissolved oxygen sensing membrane positioned over the first window and a glucose-reactive membrane positioned over the first dissolved oxygen sensing membrane, and wherein the dissolved oxygen sensing membrane provides a fluorescence response indicative of the dissolved oxygen at the first dissolved oxygen sensing membrane.

14) A sterilized enzymatic sensor assembly, including:
the enzymatic sensor of claim 1, the glucose-reactive membrane stack and the glucose-insensitive membrane stack located in a sensor tip at or near a distal end of the enzymatic sensor;
a flexible tube, the flexible tube securely connected at a proximal end to the enzymatic sensor such that a section of the enzymatic sensor extends through the interior of the flexible tube;
a distal tube cap connected to a distal end of the flexible tube, the distal tube cap comprising:
an internal passage dimensioned to allow passage of the distal tip of the enzymatic sensor therethrough;
a plurality of filtered ports in fluid communication with the internal passage; and
a gasket at or near a proximal end of the internal passage, the gasket forming a fluid-tight seal with a portion of the distal section of the enzymatic sensor extending through the gasket;
a sealing membrane occluding a distal end of the internal passage of the distal tube cap, the sealing membrane and the fluid-tight seal defining an internal storage chamber within the internal passage of the distal tube cap; and
a storage solution within the internal storage chamber to provide wet storage for the glucose-reactive membrane stack and the glucose-insensitive membrane stack.

15) A packaged sensor assembly, comprising:
a flexible package, at least a portion of the flexible package being permeable to ethylene oxide (ETO); and
a partially-assembled sensor assembly disposed within the flexible package, the partially-assembled sensor assembly comprising:
an enzymatic sensor comprising at least one enzymatic sensing membrane located at or near a distal tip of the enzymatic sensor;
a flexible tube, the flexible tube configured to be securely connected at a proximal end to the enzymatic sensor such that a distal section of the enzymatic sensor is positioned within an interior of the flexible tube;
a distal tube cap connected to a distal end of the flexible tube, the distal tube cap comprising:
an internal passage dimensioned to allow passage of the distal tip of the enzymatic sensor therethrough;
a plurality of filtered ports in fluid communication with the internal passage; and
a gasket at or near a proximal end of the internal passage, the gasket dimensioned to form a fluid-tight seal when a portion of the distal section of the enzymatic sensor is inserted through the gasket; and
a sealing membrane dimensioned to occlude a distal end of the internal passage of the distal tube cap.

16) The package of claim 15, wherein the partially-assembled sensor assembly disposed within the flexible package further comprises an aseptic connector configured to be positioned on a side of the sealing membrane opposite the distal tube cap to retain the sealing membrane in place between the distal tube cap and the aseptic connector.

17) The package of claim 16, further comprising a clamp configured to clamp the aseptic connector to the distal tube cap.

18) The package of claim 15, wherein the enzymatic sensor comprises a glucose-reactive membrane stack, and a glucose-insensitive membrane stack, wherein the enzymatic sensor is configured to provide an indication of a glucose concentration in a process medium to which the enzymatic sensor is exposed based at least in part on fluorescent responses of the glucose-reactive membrane stack and the glucose-insensitive membrane stack to illumination from a common light source.

19) A method of sterilizing a sensor assembly, the method comprising:
providing the package of claim 15;
exposing the package to ethylene oxide (ETO);
removing the ETO from the package; and
while the package remains intact, performing an initial assembly process which results in:
the sealing membrane being secured in place to occlude the distal end of the internal passage of the distal tube cap,
the interior of the flexible tube being sealed against unfiltered airflow into or out of the interior of the flexible tube; and
the enzymatic sensor being positioned within the flexible tube.

20) The method of claim 19, further comprising clamping an aseptic connector to a distal end of the distal tube cap, wherein the sealing membrane is disposed between the distal tube cap and the aseptic connector.

21) The method of claim 19, further comprising, after performing the initial assembly process:
removing the sensor assembly from the flexible package;
advancing a sensor tip of the enzymatic sensor assembly into the internal passage of the distal tube cap such that the enzymatic membranes of the sensor tip are positioned within the internal passage, and the gasket cooperates with a section of the enzymatic sensor to form a proximal end of an internal storage chamber; and
filling, using the filtered ports, the internal storage chamber with a storage solution.

22) The method of claim 19, further comprising mechanically restraining movement of the sensor tip using a restraining clip secured to the sensor assembly.
